(19) United States
(12) Patent Application Publication
(10) Pub. No.: US 2009/0215055 A1
(43) Pub. Date: Aug. 27, 2009

(54) Genetic Brain Tumor Markers

(75) Inventors: Peter James French, Rotterdam (NL); Petrus Abraham Elisa Sillevis Smitt, Rotterdam (NL)

Correspondence Address: THE WEBB LAW FIRM, P.C., 700 KOPPERS BUILDING, 436 SEVENTH AVENUE, PITTSBURGH, PA, 15219, US

(73) Assignee: ERASMUS UNIVERSITY MEDICAL CENTER ROTTERDAM, Rotterdam (NL)

(21) Appl. No.: 12/097,385

(22) PCT Filed: Dec. 13, 2005
(86) PCT No.: PCT/NL05/00855

§ 371 Date: Dec. 3, 2008

(51) Int. Cl. C12Q 1/68; C40B 40/06

(52) U.S. Cl. 435/6; 506/16

(57) ABSTRACT

The invention relates to method of genetic analysis for the prediction of treatment sensitivity and survival prognosis of patients with brain tumors, especially oligodendroglial tumors. The invention provides a method for producing a classification scheme for oligodendroglial tumors comprising the steps of a) providing a plurality of reference samples, said reference samples comprising cell samples from a plurality of reference subjects suffering from oligodendroglial tumors; b) providing reference profiles by establishing a gene expression profile for each of said reference samples individually; c) clustering said individual reference profiles according to similarity; and d) assigning an oligodendroglial tumor class to each cluster.

The invention relates to the field of diagnosis of tumors, especially brain tumors, more especially oligodendroglial tumors, more particular to the prediction of susceptibility to treatment for patients with brain tumor.

Diffuse gliomas are the most common primary central nervous system tumors in adults (Legler, J. M. et al., (1999) J. Natl. Cancer Inst. 91: 1382-1390; Macdonald, D. R. (2003) Semin. Oncol. 30: 72-76) and it is estimated that approximately 18,000 new patients per annum are diagnosed with a primary brain tumor in the USA (CBTRUS 2004-2005 statistical report). The worldwide standard for grading and classification of these tumors is at present the WHO classification (Kleihues, P. and Cavenee, W. K., World Health Organization Classification of Tumours of the Nervous System, Lyon: WHO/IARC, 2000). Based on their histological appearance gliomas can be divided into astrocytic tumors, pure oligodendroglial tumors and mixed oligoastrocytic tumors. The latter two are grouped together as oligodendroglial tumors. The oligodendrogliomas comprise approximately 20% of all gliomas, and in comparison to most other gliomas, have a relatively long average survival time (5-12 years) after diagnosis (Okamoto, Y. et al., (2004) Acta Neuropathol. 28:28; Johannesen, T. B. et al. (2003) J. Neurosurg. 99: 854-862). Two malignancy grades are recognized in oligodendrocytic tumors, Grades II (low-grade) and III (anaplastic) (Collins, V. P. (2004) J. Neurol. Neurosurg. Psych. 75 Suppl. 2: ii2-ii11).

One of the striking differences between oligodendroglial tumors and other glioma subtypes is their sensitivity to therapy, especially radiotherapy and chemotherapy. The majority of oligodendroglial tumors respond favourably to chemotherapy with alkylating agents (either temolozomide or PCV, a combination therapy of procarbazine, CCNU, and vincristine), whereas other gliomas are often chemoresistant (Van den Bent, M. J. et al. (1998) Neurology 51: 1140-1145; Van den Bent, M. J. et al. (2003) J. Clin. Oncol. 21: 2525-2528). The most favourable clinical behaviour of oligodendral tumors renders it therefore important to correctly identify this subtype of gliomas. Unfortunately, histological classification and grading of gliomas has a significant subjective component. However, malignant gliomas can also be classified according to their gene expression profile (Nutt, C. L. et al. (2003) Cancer Res. 63: 1602-1607).

In oligodendroglial tumors, there is a strong correlation between chromosomal aberrations and response to treatment (chemotherapy and/or radiotherapy). For example, a common genomic aberration is a combined loss of the short arm of chromosome 1 (1p) and the long arm of chromosome 19 (19q) (Okamoto, Y et al., 2004; Cairncross J. G. et al., (1998) J. Natl. Cancer Inst. 90:1473-1479; Kros J. M. et al., (1999) J. Pathol. 188:282-288; Smith J. S. et al., (1999) Oncogene 18:4144-4152; Thiessen B. et al., (2003) J. Neurooncol. 64:271-278; van den Bent, M. J. et al, (2003) Cancer 97:1276-1284). Loss of heterozygosity (LOH) on both chromosomal arms is correlated with a favourable response to therapy: A response to treatment is observed in 80-90% of oligodendroglial tumors with 1p LOH and in 25-30% without 1p LOH (Cairncross, J. G. et al, 1998; Thiessen, B. et al, 2003; van den Bent, M. J. et al., 2003). Other chromosomal aberrations observed at lower frequency include LOH on 10q and amplification of 7p11 (Kitange G. et al. (2004) Genes Chromosomes Cancer). These aberrations are correlated with poor prognosis and are negatively correlated with LOH on 1p and 19q. This correlation between response to treatment and chromosomal aberrations can therefore help identify chemosensitive oligodendroglial tumors. However, predicting the tumors' response to treatment by its chromosomal status also incorrectly classifies a significant percentage of tumors.

Thus, there still is a need for a more accurate prediction whether a patient with oligodendroglial tumors will be responsive to treatment and/or to predict the survival of a brain tumor patient. Expression profiling can be an alternative approach to identify oligodendroglial tumors that will benefit from therapeutic treatment. Although expression profiling has been performed on oligodendroglial tumors, mRNA expression has thus far not been correlated to treatment response.

The current inventors have now surprisingly shown that gene expression can be used to be correlated with susceptibility to treatment and increased survival, independent of the (1p and 19q) chromosomal status of the tumor. Further, also correlations have been found between gene expression and loss of 1p and 19q.

### SUMMARY OF THE INVENTION

The invention now comprise a method for producing a classification scheme for oligodendroglial tumors comprising the steps of:
a) providing a plurality of reference samples, said reference samples comprising cell samples from a plurality of reference subjects suffering from oligodendroglial tumors, with known responsiveness to therapy and survival;
b) providing reference profiles by establishing a gene expression profile, matched with parameters for sensitivity to treatment and survival for each of said reference samples individually;
c) clustering said individual reference profiles according to a statistical procedure, comprising:
   (i) K-means clustering;
   (ii) hierarchical clustering; and
   (iii) Pearson correlation coefficient analysis; and
d) assigning an oligodendroglial tumor class according to sensitivity to treatment and/or survival to each cluster.
   Specifically in such a method the clustering of said gene expression profiles is performed based on the information of differentially-expressed genes and the sensitivity to treatment and/or survival of the subject, wherein, preferably, the clustering of said gene expression profiles with respect to treatment response is performed based on the information of the genes of Table 3, whereas the clustering of said gene expression profiles with respect to survival is performed based on the information of the genes of Table 4. Another embodiment of the invention is a method for classifying an oligodendroglial tumor of a subject suffering from an glial tumor, comprising the steps of:
a) providing a classification scheme for oligodendroglial tumors according to the above described method;
b) providing a subject profile by establishing a gene expression profile for said subject;
c) clustering the subject profile together with reference profiles;
d) determining in said scheme the clustered position of said subject profile among the reference profiles, and
e) assigning to said glial tumor the oligodendroglial tumor class that corresponds to said clustered position.
   Preferably herein the gene expression profile with respect to treatment response comprises the expression parameters of a set of genes according to table 3, still more preferably 1 to 50 genes of the genes of table 3, whereas the gene expression profile with respect to survival comprises the expression parameters of a set of genes according to Table 4, more preferably 1 tot 50 genes of the genes of Table 4. A further embodiment of the invention is a method of determining the prognosis for a subject suffering from an oligodendroglial tumor, said method comprising the steps of:
a) providing a classification scheme for oligodendroglial tumors by producing such a scheme according to the above described method;
b) determining the prognosis for each olidendroglial tumor class in said scheme based on clinical records for the subjects comprised in said class;
c) establishing the oligodendroglial class of a subject suffering from an oligodendroglial tumor by classifying the oligodendroglial tumor in said subject according to a method according to the invention, and
d) assigning to said subject the prognosis corresponding to the established oligodendroglial tumor class of said subject.
   Alternatively, the invention provides for a method of determining the prognosis for a subject suffering from an oligodendroglial tumor, said method comprising the steps of:
a) isolation of RNA from tumor cells of said subject;
b) preparation of antisense, biotinylated RNA to the RNA of step a);
c) hybridisation of said antisense, biotinylated DNA on Affymetrix U133A or U133 Plus2.0 GeneChips®;
d) normalising the measured values for the gene set of Table 3;
e) clustering the obtained data together with reference data, obtained from a reference set of patients with known prognoses; and
f) determining the prognosis on basis of the subgroup/cluster to which the data of the subject are clustering.

In another embodiment, the invention provides for an oligonucleotide microarray of maximal 500 probesets, comprising at least 1, preferably at least 2, more preferably at least 25, still more preferably at least 100 oligonucleotide probes which each are capable of hybridizing under stringent conditions to different genes of the oligodendroglial tumor-associated genes selected from Table 3. Alternatively, the invention provides for an oligonucleotide microarray of maximal 500 probesets, comprising at least 1, preferably at least 2, more preferably at least 25, still more preferably at least 100 oligonucleotide probes which each are capable of hybridizing under stringent conditions to different genes of the oligodendroglial tumor-associated genes selected from Table 4.

In oligodendrogliomas there is a strong correlation between LOH on 1p/19q and response to treatment. In another embodiment, the invention provides for a method using an oligonucleotide microarray, which can be used for the determination of the presence of 1p LOH, 19q LOH or 1p/19q LOH. Particularly, the microarray for these determination should comprise the genesets of Table 5, 6 and 7, respectively. Accordingly, the invention also comprises an oligonucleotide microarray of maximal 500 probesets, comprising at least 1, preferably at least 2, more preferably at least 25, still more preferably at least 50 oligonucleotide probes which each are capable of hybridizing under stringent conditions to different genes of the oligodendroglial tumor-associated genes selected form Table 5, 6 and 7, respectively.

For the above described methods, the invention also comprises a kit-of-parts comprising an oligonucleotide microarray as described above and means for comparing a gene expression profile determined by using said microarray with a database of oligodendroglial tumor reference expression profiles.

### LEGENDS TO THE FIGURES

FIG. 1.

Correlation plot of all samples. Samples are plotted against each other to determine the degree of similarity based on expressed genes. Red and blue denote high and low similarity respectively (scale bar). Below the correlation plot is a graphic representation of histological and patient data. Tissue: origin of sample [symbol] control cortex, [symbol] control white matter, [symbol] low-grade oligodendroglioma, [symbol] anaplastic oligodendroglioma. 1p, 19q, 10q LOH: [symbol] no LOH, [symbol] LOH. (LOH: loss of heterozygosity). EGFR ampl: amplification of the EFGR chromosomal locus: [symbol] no amplification, [symbol] amplification, Response: response to therapy complete [symbol] response, [symbol] partial response, [symbol] stable disease, [symbol] progressive disease. Surv tot: survival (years) from time of diagnosis. [symbol] >10, [symbol] 7-10, [symbol] 3-7, [symbol] <3.A: patient alive at time of analysis.

FIG. 2.

Principle components analysis (PCA) and hierarchical clustering of 60 probesets differentially expressed between oligodendroglial tumors with combined 1p and 19q LOH and those that have retained both 1p and 19q arms. A: samples are separated on their 1p and 19q chromosomal status by the first principle component axis (PCA1) whereas PCA2 separates control brain from anaplastic oligodendroglial tumors. The 1p and 19q status are color coded with [symbol] =no LOH on 1p and 19q, [symbol] =LOH on 1p and 19q, and [symbol] LOH on either 1p or 19q. B: Hierarchical clustering shows relative expression levels of individual genes (columns) plotted against individual tumor samples (rows). For clarity, control brain samples were omitted from the clustering analysis. Gene expression levels are color coded with red and green indicating high (+2) and low green (−2) expression respectively (on a log 2 scale). Dendrograms denote hierarchical clustering (Euclidian distance) of samples (top) and genes (left). The 1p and 19q status in indicated below the hierarchical clustering ( [symbol] M=no LOH, [symbol] =LOH). As can be seen, hierarchical clustering clearly identifies two main subgroups associated with 1p/19q LOH.

FIG. 3.

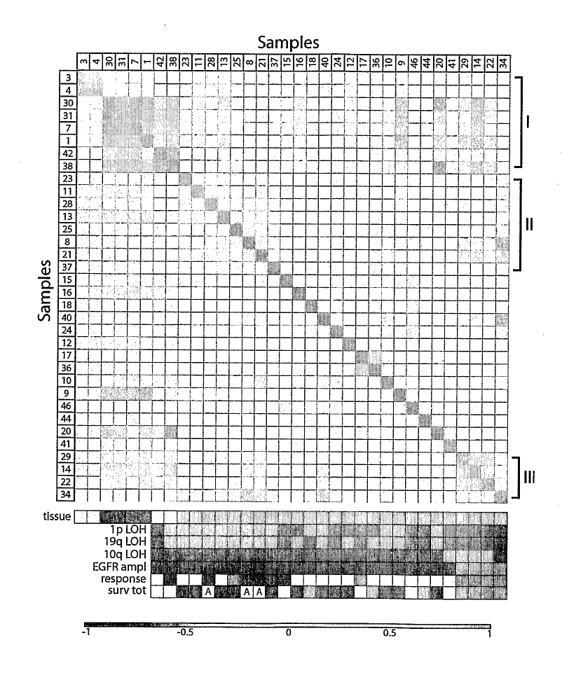

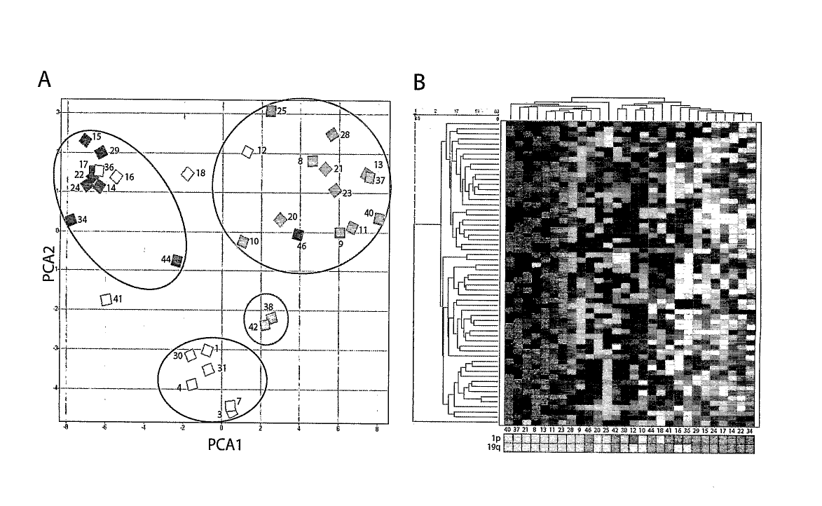

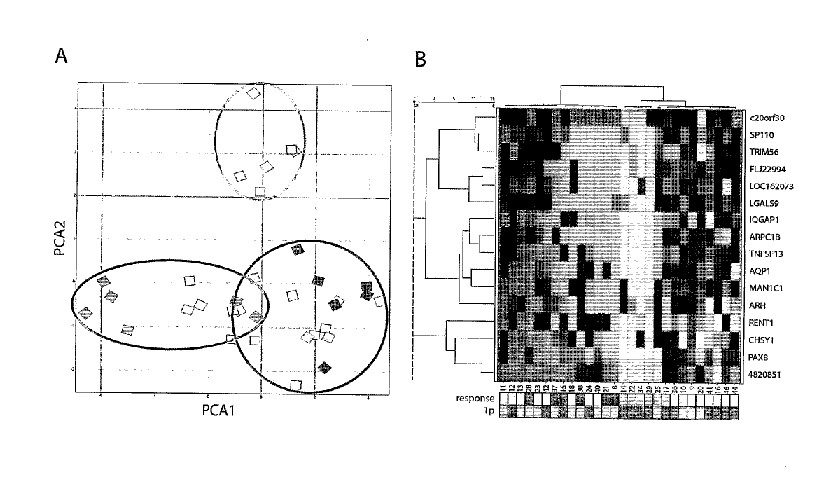

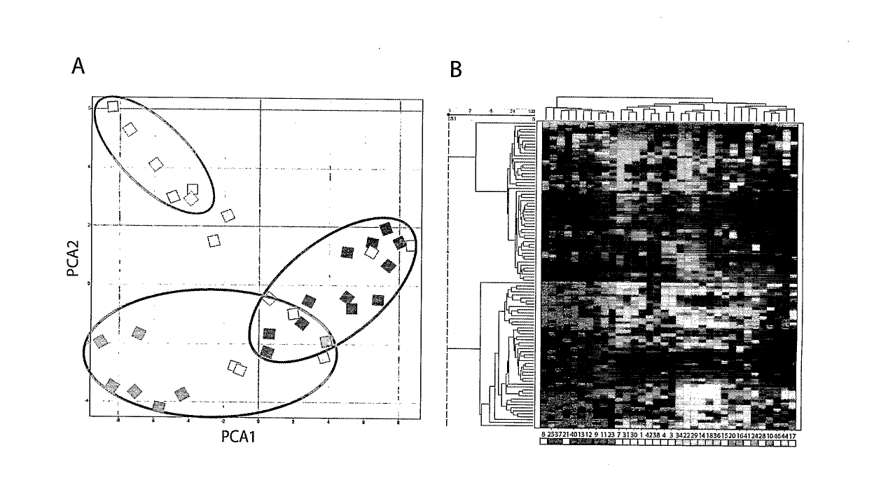

PCA and hierarchical clustering based on 16 probesets differentially expressed between chemosensitive (CR+PR (complete response, partial response)) and chemoresistant (SD+PD, stable disease, progressive disease)) oligodendroglial tumors. A: samples are separated on their response to chemotherapy by the first principle component axis (PCA1) whereas PCA2 separates control brain from anaplastic oligodendroglial tumors. B: Hierarchical clustering based on 16 differentially expressed probesets. Relative expression levels of individual genes (columns) are plotted against individual tumor samples (rows). Gene expression levels are color coded with red and green indicating high (+1.8) and low green (−1.8) expression respectively. Dendrograms denote hierarchical clustering of samples (top) and genes (left) using Wards method. Hierarchical clustering separates tumors that fully respond to chemotherapy (CR) from tumors that do not respond (SD+PD). Furthermore, hierarchical clustering also clearly separates tumors with poor prognosis (subgroup III in FIG. 1) from other oligodendroglial tumors. Responses in oligodendroglial tumors are color coded with [symbol] complete response, [symbol] partial response, [symbol] stable disease, [symbol] progressive disease, [symbol] control brain. 1p chromosomal status is depicted as [symbol] no loss of 1p and [symbol] 1p LOH.

FIG. 4.

PCA hierarchical clustering based on 103 probesets associated with survival after diagnosis. A: PCA identifies three main clusters of samples: oligodendroglial tumors with short survival, oligodendroglial tumors with long survival and control samples. Two low-grade samples (38 and 42, survival <10 years [symbol] ) cluster between control and tumor samples. PCA analysis separates short vs. long survivors on the first principle component axis (PCA1) whereas control and tumor samples are separated by the second PCA axis. B: Hierarchical clustering based on 103 differentially expressed probesets. Relative expression levels of individual genes (columns) are plotted against individual tumor samples (rows). Gene expression levels are color coded with red and green indicating high (+2) and low green (−2) expression respectively. Dendrograms denote hierarchical clustering (Euclidian distance) of samples (top) and genes (left). Interestingly, the subgroups identified by hierarchical clustering are virtually identical to the subgroups that were identified by unsupervised clustering (FIG. 1). Survival after diagnosis is depicted as [symbol] >10 years survival, [symbol] <10 years survival, [symbol] <7 years survival, [symbol] <4 years survival, [symbol] patient still alive or, [symbol] control brain.

### DETAILED DESCRIPTION OF THE INVENTION

The current inventors performed expression profiling on oligodendroglial tumors and correlated the results to response to treatment, survival after diagnosis and common chromosomal aberrations. One of the findings was that the chromosomal aberrations led to ˜50% expression of some but not all of the genes that had been deleted, Thus, this means that it is not straightforward to use the expression data of the genes from the 1p and 19q loci for the determination of the presence of a loss of heterozygosity (LOH) in these areas. Yet, the present inventors have found that a subset of genes, which show a reduced expression when one of the chromosomal arms 1p and 19q are deleted can be used to detect these chromosomal aberrations. The genes, which can distinguish between the presence or absence of 1p have been listed in Table 5, for LOH of 19q the genes are listed in Table 6, and Table 7 gives the list of discriminating genes for combined 1p and 19q LOH.

This means that gene expression data can be used for the determination of LOH of 1p and/or 19q. This is advantageous, since currently for said determination a FISH (Fluorescence In Situ Hybridisation) or LOH (loss of heterozygosity)-PCR is used, which are specialised tests, using labelled probes. Now it has been established that a similar determination can be achieved by using standard array technology.

Further, the present study shows that the currently used predictions, based on loss of 1p, were only correctly assigned to the correct treatment response group in 20/28 (71%) of the cases, both because of positive and negative misclassifications

The term “classifying” is used in its art-recognized meaning and thus refers to arranging or ordering items, i.c. gene expression profiles, by classes or categories or dividing them into logically hierarchical classes, subclasses, and sub-subclasses based on the characteristics they have in common and/or that distinguish them. In particular “classifying” refers to assigning, to a class or kind, an unclassified item. A “class” then being a grouping of items, based on one or more characteristics, attributes, properties, qualities, effects, parameters, etc., which they have in common, for the purpose of classifying them according to an established system or scheme.

The term “classification scheme” is used in its art-recognized meaning and thus refers to a list of classes arranged according to a set of pre-established principles, for the purpose of organizing items in a collection or into groups based on their similarities and differences.

The term “clustering” refers to the activity of collecting, assembling and/or uniting into a cluster or clusters items with the same or similar elements, a “cluster” referring to a group or number of the same or similar items, i.c. gene expression profiles, gathered or occurring closely together based on similarity of characteristics. “Clustered” indicates an item has been subjected to clustering.

The term “clustered position” refers to the location of an individual item, i.c. a gene expression profile, in amongst a number of clusters, said location being determined by clustering said item with at least a number of items from known clusters.

The process of clustering used in a method of the present invention may be any mathematical process known to compare items for similarity in characteristics, attributes, properties, qualities, effects, parameters, etc. Statistical analysis, such as for instance multivariance analysis, or other methods of analysis may be used. Preferably methods of analysis such as self-organising maps, hierarchical clustering, multidimensional scaling, principle component analysis, supervised learning, k-nearest neighbours, support vector machines, discriminant analysis, partial least square methods and/or Pearson's correlation coefficient analysis are used. In another preferred embodiment of a method of the present invention Pearson's correlation coefficient analysis, significance analysis of microarrays (SAM) and/or prediction analysis of microarrays (PAM) are used to cluster gene expression profiles according to similarity. A highly preferred method of clustering comprises similarity clustering of gene expression profiles wherein the expression level of differentially-expressed genes, having markedly lower or higher expression than the geometric mean expression level determined for all genes in all profiles to be clustered, is log(2) transformed, and wherein the transformed expression levels of all differentially-expressed genes in all profiles to be clustered is clustered by using K-means. A numerical query may then be used to select a subset of genes used in the process of hierarchical clustering (Eisen et al., 1998), thus, numerical queries may be run to select differentially expressed genes relative to the calculated geometric mean to select a smaller group of genes for hierarchical clustering.

Unsupervised sample clustering using genes obtained by numerical or threshold filtering is used to identify discrete clusters of samples as well as the gene-signatures associated with these clusters. The term gene signatures is used herein to refer to the set of genes that define the discrete position of the cluster apart from all other clusters, and includes cluster-specific genes. A numerical or threshold filtering is used to select genes for the analysis that are most likely of diagnostic relevance. Hierarchical clustering allows for visualization of large variation in gene expression across samples or present in most samples, and these genes could be used for unsupervised clustering so that clustering results are not affected by the noise from absent or non-changed genes.

Thus, while K-means clustering may be performed on all genes, the Pearson correlation is preferably calculated based on a subset of genes. Generally speaking the larger the threshold for accepting a deviation or change from the geometric mean, the smaller the number of genes that is selected by this filtering procedure. Different cut-off or threshold values were used to prepare lists with different numbers of genes. The higher the number of genes selected and included on such lists, the more noise is generally encountered within the dataset, because there will be a relatively large contribution of non-tumor pathway related genes in such lists. The filtering and selection procedure is preferably optimized such that the analysis is performed on as many genes as possible, while minimizing the noise.

All genes with changed expression values in at least one sample higher than or equal to 1.5 times the log(2) transformed expression values and genes with changed expression values lower than or equal to −1.5 times the log(2) transformed expression value means are selected for unsupervised clustering.

The subset of genes showing a markedly higher or lower expression than the geometric mean may for instance be a value that is more than 1.5 times the geometric mean value, preferably more than 2 times the geometric mean value, Likewise, a markedly lower expression than the geometric mean expression level may for instance be a value that is less than 0.8 times the geometric mean value, preferably less than 0.6 times the geometric mean value.

Independently (see FIG. 1) a Pearson correlation coefficient analysis was performed on the samples (1881 probesets), which showed that clustering of patients is feasible.

The present invention now provides several methods to accurately identify known as well as newly discovered diagnostically, prognostically and therapeutically relevant subgroups of oligodendroglial tumors, as well as methods that can predict if treatment is likely to be effective. The basis of these methods resides in the measurement of (oligodendroglial tumor-specific) gene expression in subjects suffering from brain tumors. The methods and compositions of the invention thus provide tools useful in choosing a therapy for brain tumor patients, including methods for assigning an brain tumor patient to a brain tumor class or cluster, methods of choosing a therapy for a brain tumor patient, and methods of determining the survival prognosis for a brain tumor patient.

The methods of the invention comprise in various aspects the steps of establishing a gene expression profile of subject samples, for instance of reference subjects suffering from a brain tumor or of a subject diagnosed or classified as having a brain tumor. The expression profiles of the present invention are generated from samples from subjects having a brain tumor. The samples from the subject used to generate the expression profiles of the present invention can be derived from a tumor biopsy, wherein the sample comprises preferably more than 75% tumor cells.

“Gene expression profiling” or “expression profiling” is used herein in its art-recognised meaning and refers to a method for measuring the transcriptional state (mRNA) or the translational state (protein) of a plurality of genes in a cell. Depending on the method used, such measurements may involve the genome-wide assessment of gene expression, but also the measurement of the expression level of selected genes, resulting in the establishment of a “gene expression profile” or “expression profile”, which terms are used in that meaning hereinbelow. As used herein, an “expression profile” comprises one or more values corresponding to a measurement of the relative abundance of a gene expression product. Such values may include measurements of RNA levels or protein abundance. Thus, the expression profile can comprise values representing the measurement of the transcriptional state or the translational state of the gene. In relation thereto, reference is made to U.S. Pat. Nos. 6,040,138, 5,800,992, 6,020,135, 6,344,316, and 6,033,860.

The transcriptional state of a sample includes the idensities and relative abundance of the RNA species, especially mRNAs present in the sample. Preferably, a substantial fraction of all constituent RNA species in the sample are measured, but at least a sufficient fraction to characterize the transcriptional state of the sample is measured. The transcriptional state can be conveniently determined by measuring transcript abundance by any of several existing gene expression technologies.

Translational state includes the identities and relative abundance of the constituent protein species in the sample. As is known to those of skill in the art, the transcriptional state and translational state are often related.

Each value in the expression profiles as determined and embodied in the present invention is a measurement representing the absolute or the relative expression level of a differentially-expressed gene. The expression levels of these genes may be determined by any method known in the art for assessing the expression level of an RNA or protein molecule in a sample. For example, expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, to which explicit reference is made. The gene expression monitoring system may also comprise nucleic acid probes in solution.

In one embodiment of the invention, microarrays are used to measure the values to be included in the expression profiles. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, the Experimental section. See also, U.S. Pat. Nos. 6,040,138, 5,800,992 and 6,020,135, 6,033,860, and 6,344,316, to which explicit reference is made. High-density oligonucleotide arrays are particularly useful for determining the gene expression profile for a large number of RNA's in a sample.

In one approach, total RNA isolated from the sample is converted to labeled cRNA and then hybridized to an oligonucleotide array. Each sample is hybridized to a separate array. Relative transcript levels are calculated by reference to appropriate controls present on the array and in the sample. See, for example, the Experimental section.

In another embodiment, the values in the expression profile are obtained by measuring the abundance of the protein products of the differentially-expressed genes. The abundance of these protein products can be determined, for example, using antibodies specific for the protein products of the differentially-expressed genes. The term “antibody” as used herein refers to an immunoglobulin molecule or immunologically active portion thereof, i.e., an antigen-binding portion. Examples of immunologically active portions of immunoglobulin molecules include F(ab) and F(ab′)2 fragments which can be generated by treating the antibody with an enzyme such as pepsin. The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent. A full-length protein product from a differentially-expressed gene, or an antigenic peptide fragment of the protein product can be used as an immunogen. Preferred epitopes encompassed by the antigenic peptide are regions of the protein product of the differentially-expressed gene that are located on the surface of the protein, e.g., hydrophilic regions, as well as regions with high antigenicity. The antibody can be used to detect the protein product of the differentially-expressed gene in order to evaluate the abundance and pattern of expression of the protein. These antibodies can also be used diagnostically to monitor protein levels in tissue as part of a clinical testing procedure, e.g., to, for example, determine the efficacy of a given therapy. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, (3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, quantum dots or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Once the values comprised in the subject expression profile and the reference expression profile or expression profiles are established, the subject profile is compared to the reference profile to determine whether the subject expression profile is sufficiently similar to the reference profile. Alternatively, the subject expression profile is compared to a plurality of reference expression profiles to select the reference expression profile that is most similar to the subject expression profile. Any method known in the art for comparing two or more data sets to detect similarity between them may be used to compare the subject expression profile to the reference expression profiles. In some embodiments, the subject expression profile and the reference profile are compared using a supervised learning algorithm such as the support vector machine (SVM) algorithm, prediction by collective likelihood of emerging patterns (PCL) algorithm, the k-nearest neighbour algorithm, or the Artificial Neural Network algorithm. To determine whether a subject expression profile shows “statistically significant similarity” or “sufficient similarity” to a reference profile, statistical tests may be performed to determine whether the similarity between the subject expression profile and the reference expression profile is likely to have been achieved by a random event. Any statistical test that can calculate the likelihood that the similarity between the subject expression profile and the reference profile results from a random event can be used. The accuracy of assigning a subject to an oligodendroglial tumor class based on similarity between differentially-expressed genes is affected largely by the heterogeneity within the patient population, as is reflected by the deviation from the geometric mean. Therefore, when more accurate diagnoses are required, the stringency in evaluating the similarity between the subject and the reference profile should be increased by changing the numerical query.

The method used for comparing a subject expression profile to one or more reference profiles is preferably carried out by re-running the subsequent analyses in a (n+1) modus by performing clustering methods as described herein. Also, in order to identify the oligodendroglial tumor class reference profile that is most similar to the subject expression profile, as performed in the methods for establishing the oligodendroglial tumor class of a subject having a brain tumor, i.e. by diagnosing presence of an oligodendroglial tumor in a subject or by classifying the oligodendroglial tumor in a subject, profiles are clustered according to similarity and it is determined whether the subject profile corresponds to a known class of reference profiles. In assigning a subject oligodendroglial tumor to a specific oligodendroglial tumor class for instance, this method is used wherein the clustered position of the subject profile, obtained after performing the clustering analysis of the present invention, is compared to any known oligodendroglial tumor class. If the clustered position of the subject profile is within a cluster of reference profiles, i.e. forms a cluster therewith after performing the similarity clustering method, it is said that the oligodendroglial tumor of the subject corresponds to the oligodendroglial tumor class of reference profiles.

In some embodiments of the present invention, the expression profiles comprise values representing the expression levels of genes that are differentially-expressed in oligodendroglial tumor classes. The term “differentially-expressed” as used herein means that the measured expression level of a particular gene in the expression profile of one subject differs at least n-fold from the geometric mean calculated from all patient profiles. The expression level may be also be up-regulated or down-regulated in a sample from a subject in comparison with a sample from a normal brain sample, or in comparison with the mean of all oligodendroglial tumor patients. Examples of genes that are differentially expressed in brain tumor patients which respond to therapy and brain tumor patients which do not respond to therapy, short vs. long survivors and 1p and/or 19q LOH vs. no loss are listed in Tables 3, 4, 5, 6 and 7.

It should be noted that many genes will occur, of which the measured expression level differs at least n-fold from the geometric mean expression level for that gene of all reference profiles. This may for instance be due to the different physiological state of the measured cells, to biological variation or to the presence of other diseased states. Therefore, the presence of a differentially-expressed gene is not necessarily informative for determining the presence of different oligodendroglial tumor classes, nor is every differentially-expressed gene suitable for performing diagnostic tests. Moreover, a cluster-specific differential gene expression, as defined herein, is most likely to be informative only in a test among subjects having brain tumors. Therefore, a diagnostic test performed by using cluster-specific gene detection should preferably be performed on a subject in which the presence of an oligodendroglial tumor is confirmed. This confirmation may for instance be obtained by standard macroscopic and microscopic detection methods.

The present invention provides groups of genes that are differentially-expressed in diagnostic oligodendroglial tumor biopsy and surgical resection samples of patients in different therapeutic groups (i.e. responders/non-responders, or short-survivors/long-survivors). Values representing the expression levels of the nucleic acid molecules detected by the probes were analyzed as described in the Experimental section using Omniviz and SAM analysis tools. Omniviz software was used to perform all clustering steps such as K-means, Hierarchical and Pearson correlation tests. SAM was used specifically to identify the genes underlying the clinically relevant groups identified in the Pearson correlation analysis. PAM is used to decide the minimum number of genes necessary to diagnose all individual patients within the given groups of the Pearson correlation.

In short, expression profiling was carried out on biopsy material from 28 brain tumor patients. Unsupervised clustering was used to identify novel (sub)groups within the Pearson correlation following the hierarchical clustering. After running the SAM analysis the diagnostic gene-signatures (incl. cluster-specific genes) were obtained.

It appeared that a clustering separating the different groups of patients could be performed on the basis of differential expression of a plurality of genes.

The present invention thus provides a method of classifying oligodendroglial tumors. Using this method, a total of 28 brain tumor samples analysed on a DNA microarray consisting of 54675 probe sets, representing approximately 23000 genes, could be classified. The classification into patient groups was performed on the basis of strong correlation between their individual differential expression profiles within a group for 1881 probe sets (˜1413 genes). The methods used to analyze the expression level values to identify differentially-expressed genes were employed such that optimal results in clustering, i.e. unsupervised ordering, were obtained. The genes that defined the position or clustering of these patient groups could be determined and the minimal sets of genes required to accurately predict the prognostically important classes could be derived. It should be understood that the method for classifying oligodendroglial tumors according to the present invention may result in a distinct pattern and therefore in a different classification scheme when other (numbers of) subjects are used as reference, or when other types of oligonucleotide microarrays for establishing gene expression profiles are used.

The present invention thus provides a comprehensive classification of oligodendroglial tumors covering previously identified therapeutically defined classes. Further analysis of classes by significance analysis of microarrays (SAM) to determine the minimum number of genes that defined or predicted these classes resulted in the establishment of cluster-specific genes or signature genes.

The methods of the present invention comprise in some aspects the step of defining cluster-specific genes by selecting those genes of which the expression level characterizes the clustered position of the corresponding oligodendroglial tumor class within a classification scheme of the present invention. Such cluster-specific genes are selected preferably on the basis of SAM analysis. This method of selection comprises the following.

The methods of the present invention comprise in some aspects the step of establishing whether the level of expression of cluster-specific genes in a subject shares sufficient similarity to the level of expression that is characteristic for an individual oligodendroglial tumor class. This step is necessary in determining the presence of that particular oligodendroglial tumor class in a subject under investigation, in which case the expression of that gene is used as a prognostic marker. Whether the level of expression of cluster-specific genes in a subject shares sufficient similarity to the level of expression of that particular gene in an individual oligodendroglial tumor class may for instance be determined by setting a threshold value.

The present invention also reveals genes with a high differential level of expression in specific oligodendroglial tumor classes compared to the geometric mean of all reference subjects. These highly differentially-expressed genes are selected from the genes shown in Tables 3-7, These genes and their expression products are useful as markers to predict the responsiveness to treatment, 1p and/or 19q loss of heterozygosity or survival chance in a patient. Antibodies or other reagents or tools may be used to detect the presence of these markers of brain tumor.

The present invention also reveals gene expression profiles comprising values representing the expression levels of genes in the various identified oligodendroglial tumor classes. In a preferred embodiment, these expression profiles comprise the values representing the differential expression levels. Thus, in one embodiment the expression profiles of the invention comprise one or more values representing the expression level of a gene having differential expression in a defined oligodendroglial tumor class. Each expression profile contains a sufficient number of values such that the profile can be used to distinguish treatment response groups, to distinguish groups with different survival, an to distinguish groups with 1p and/or 19q LOH. The expression profile comprises more than one or two values corresponding to a differentially-expressed gene, for example at least 3 values, at least 4 values, at least 5 values, at least 6 values, at least 7 values, at least 8 values, at least 9 values, at least 10 values, at least 11 values, at least 12 values, at least 13 values, at least 14 values, at least 15 values, at least 16 values, at least 17 values, at least 18 values, at least 19 values, at least 20 values, at least 22 values, at least 25 values, at least 27 values, at least 30 values, at least 35 values, at least 40 values, at least 45 values, at least 50 values, at least 75 values, at least 100 values, at least 125 values, at least 150 values, at least 175 values, at least 200 values, at least 250 values, at least 300 values, at least 400 values, at least 500 values, at least 600 values, at least 700 values, at least 800 values, at least 900 values, at least 1000 values, at least 1200 values, at least 1500 values, or at least 2000 or more values.

It is recognized that the diagnostic accuracy of assigning a subject to an oligodendroglial tumor class will vary based on the number of values contained in the expression profile. Generally, the number of values contained in the expression profile is selected such that the diagnostic accuracy is at least 85%, at least 87%, at least 90%, at least 91%, at least 92%, at least 93%, at least 94%, at least 95%, at least 96%, at least 97%, at least 98%, or at least 99%, as calculated using methods described elsewhere herein, with an obvious preference for higher percentages of diagnostic accuracy.

It is recognized that the diagnostic accuracy of assigning a subject to an oligodendroglial tumor class will vary based on the strength of the correlation between the expression levels of the differentially-expressed genes within that specific oligodendroglial tumor class. When the values in the expression profiles represent the expression levels of genes whose expression is strongly correlated with that specific oligodendroglial tumor class, it may be possible to use fewer number of values (genes) in the expression profile and still obtain an acceptable level of diagnostic or prognostic accuracy.

The strength of the correlation between the expression level of a differentially-expressed gene and a specific oligodendroglial tumor class may be determined by a statistical test of significance. For example, the chi square test used to select genes in some embodiments of the present invention assigns a chi square value to each differentially-expressed gene, indicating the strength of the correlation of the expression of that gene to a specific oligodendroglial tumor class. Similarly, the T-statistics metric and the Wilkins' metric both provide a value or score indicative of the strength of the correlation between the expression of the gene and its specific oligodendroglial tumor class. These scores may be used to select the genes of which the expression levels have the greatest correlation with a particular oligodendroglial tumor class to increase the diagnostic or prognostic accuracy of the methods of the invention, or in order to reduce the number of values contained in the expression profile while maintaining the diagnostic or prognostic accuracy of the expression profile. Preferably, a database is kept wherein the expression profiles of reference subjects are collected and to which database new profiles can be added and clustered with the already existing profiles such as to provide the clustered position of said new profile among the already present reference profiles. Furthermore, the addition of new profiles to the database will improve the diagnostic and prognostic accuracy of the methods of the invention. Preferably, in a method of the present invention SAM or PAM analysis tools are used to determine the strength of such correlations.

The methods of the invention comprise the steps of providing an expression profile from a sample from a subject affected by oligodendroglial tumor and comparing this subject expression profile to one or more reference profiles that are associated with a particular oligodendroglial tumor class with a known prognosis, or a class with a favourable response to therapy. By identifying the oligodendroglial tumor class reference profile that is most similar to the subject expression profile, e.g. when their clustered positions fall together, the subject can be assigned to an oligodendroglial tumor class. The oligodendroglial class assigned is that with which the reference profile(s) are associated. Similarly, the prognosis of a subject affected by an oligodendroglial tumor can be predicted by determining whether the expression profile from the subject is sufficiently similar to a reference profile associated with an established prognosis, such as a good prognosis or a bad prognosis. Whenever a subject's expression profile can be assigned to one of the reference profile(s), a preferred intervention strategy, or therapeutic treatment can then be proposed for said subject, and said subject can be treated according to said assigned strategy. As a result, treatment of a subject with an oligodendroglial can be optimized according to the identified cluster.

In one aspect, the present invention provides a method of determining the prognosis for a brain tumor patient, said method comprising the steps of providing a classification scheme for oligodendroglial tumors by producing such a scheme according to a method of the invention for reference subjects having known post-therapy lifetimes. The present invention provides for the assignment of the various clinical data recorded to reference subjects affected by brain tumors. This assignment preferably occurs in a database. This has the advantage that once a new subject is identified as belonging to a particular oligodendroglial tumor class, then the prognosis that is assigned to that class may be assigned to that subject.

The present invention provides compositions that are useful in determining the gene expression profile for a subject affected by an oligodendroglial tumor and selecting a reference profile that is similar to the subject expression profile. These compositions include arrays comprising a substrate having capture probes that can bind specifically to nucleic acid molecules that are differentially-expressed in oligodendroglial tumor classes. Also provided is a computer-readable medium having digitally encoded reference profiles useful in the methods of the claimed invention.

The present invention provides arrays comprising capture probes for detection of polynucleotides (transcriptional state) or for detection of proteins (translational state) in order to detect differentially-expressed genes of the invention. By “array” is intended a solid support or substrate with peptide or nucleic acid probes attached to said support or substrate. Arrays typically comprise a plurality of different nucleic acid or peptide capture probes that are coupled to a surface of a substrate in different, known locations. These arrays, also described as “microarrays” or colloquially “chips” have been generally described in the art, and reference is made U.S. Pat. Nos. 5,143,854, 5,445,934, 5,744,305, 5,677,195, 6,040,193, 5,424,186, 6,329,143, and 6,309,831 and Fodor et al. (1991) Science 251:767-77. These arrays may generally be produced using mechanical synthesis methods or light directed synthesis methods which incorporate a combination of photolithographic methods and solid phase synthesis methods. Typically, “oligonucleotide microarrays” will be used for determining the transcriptional state, whereas “peptide microarrays” will be used for determining the translational state of a cell.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogues can be made; alternatively, mixtures of different nucleic acid analogues, and mixtures of naturally occurring nucleic acids and analogues may be made.

Particularly preferred are peptide nucleic acids (PNA) which includes peptide nucleic acid analogues. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

“Transcript” typically refers to a naturally occurring RNA, e.g., a pre-mRNA, hnRNA, or mRNA. As used herein, the term “nucleoside” includes nucleotides and nucleoside and nucleotide analogues, and modified nucleosides such as amino modified nucleosides. In addition, “nucleoside” includes non-naturally occurring analogue structures. Thus, e.g. the individual units of a peptide nucleic acid, each containing a base, are referred to herein as a nucleoside.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled such as with isotopes, chromophores, lumiphores, chromogens, or indirectly labeled such as with biotin to which a streptavidin complex may later bind or with enzymatic labels. By assaying for the hybridization of the probe to its target nucleic acid sequence, one can detect the presence or absence of the select sequence or subsequence. Diagnosis or prognosis may be based at the genomic level, or at the level of RNA or protein expression.

The skilled person is capable of designing oligonucleotide probes that can be used in diagnostic methods of the present invention. Preferably, such probes are immobilised on a solid surface as to form an oligonucleotide microarray of the invention. The oligonucleotide probes useful in methods of the present invention are capable of hybridizing under stringent conditions to oligodendroglial tumor-associated nucleic acids, such as to one or more of the genes selected from Table 2 or Table 3.

Techniques for the synthesis of arrays using mechanical synthesis methods are described in, e.g., U.S. Pat. No. 5,384,261, to which reference is made herein. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, for the purpose of which reference is made to U.S. Pat. Nos. 5,770,358, 5,789,162, 5,708,153, 6,040,193 and 5,800,992. Arrays may be packaged in such a manner as to allow for diagnostics or other manipulation of an all-inclusive device. Reference is for example made to U.S. Pat. Nos. 5,856,174 and 5,922,591.

The arrays provided by the present invention comprise capture probes that can specifically bind a nucleic acid molecule that is differentially-expressed in oligodendroglial tumor classes. These arrays can be used to measure the expression levels of nucleic acid molecules to thereby create an expression profile for use in methods of determining the therapeutic treatment and prognosis for oligodendroglial tumor patients.

In some embodiments, each capture probe in the array detects a nucleic acid molecule selected from the nucleic acid molecules designated in Tables 2 or Table 3. The designated nucleic acid molecules include those differentially-expressed in oligodendroglial tumor classes.

The arrays of the invention comprise a substrate having a plurality of addresses, where each address has a capture probe that can specifically bind a target nucleic acid molecule. The number of addresses on the substrate varies with the purpose for which the array is intended. The arrays may be low-density arrays or high-density arrays and may contain 4 or more, 8 or more, 12 or more, 16 or more, 20 or more, 24 or more, 32 or more, 48 or more, 64 or more, 72 or more 80 or more, 96, or more addresses, or 192 or more, 288 or more, 384 or more, 768 or more, 1536 or more, 3072 or more, 6144 or more, 9216 or more, 12288 or more, 15360 or more, or 18432 or more addresses. In some embodiments, the substrate has no more than 12, 24, 48, 96, or 192, or 384 addresses, no more than 500, 600, 700, 800, or 900 addresses, or no more than 1000, 1200, 1600, 2400, or 3600 addresses.

The invention also provides a computer-readable medium comprising one or more digitally encoded expression profiles, where each profile has one or more values representing the expression of a gene that is differentially-expressed in an oligodendroglial tumor class. The preparation and use of such profiles is well within the reach of the skilled person (see e.g. WO 03/083140). In some embodiments, the digitally-encoded expression profiles are comprised in a database. See, for example, U.S. Pat. No. 6,308,170.

The present invention also provides kits useful for predicting the responsiveness to therapy in subjects affected by an oligodendroglial tumor. These kits comprise an array and a computer readable medium. The array comprises a substrate having addresses, where each address has a capture probe that can specifically bind a nucleic acid molecule (by using an oligonucleotide array) or a peptide (by using a peptide array) that is differentially-expressed in an oligodendroglial tumor class. The results are converted into a computer-readable medium that has digitally-encoded expression profiles containing values representing the expression level of a nucleic acid molecule detected by the array.

By using the array described above, the amounts of various kinds of nucleic acid molecules contained in a nucleic acid sample can be simultaneously determined. In addition, there is an advantage such that the determination can be carried out even with a small amount of the nucleic acid sample. For instance, mRNA in the sample is labeled, or labeled cDNA is prepared by using mRNA as a template, and the labeled mRNA or cDNA is subjected to hybridization with the array, so that mRNAs being expressed in the sample are simultaneously detected, whereby their expression levels can be determined.

Genes each of which expression is altered due to an oligodendroglial tumor can be found by determining expression levels of various genes in the tumor cells and classified into certain types as described above and comparing the expression levels with the expression level in a control tissue.

The method for determining the expression levels of genes is not particularly limited, and any of techniques for confirming alterations of the gene expressions mentioned above can be suitably used. Among all, the method using the array is especially preferable because the expressions of a large number of genes can be simultaneously determined. Suitable arrays are commercially available, e.g., from Affymetrix.

For instance, mRNA is prepared from tumor cells, and then reverse transcription is carried out with the resulting mRNA as a template. During this process, labeled cDNA can be obtained by using, for instance, any suitable labeled primers or labeled nucleotides.

As to the labeling substance used for labeling, there can be used substances such as radioisotopes, fluorescent substances, chemiluminescent substances and substances with fluophor, and the like. For instance, the fluorescent substance includes Cy2, Fluor X, Cy3, Cy3.5, Cy5, Cy5.5, Cy7, fluorescein isothiocyanate (FITC), Texas Red, Rhodamine and the like. In addition, it is desired that samples to be tested (cancer samples to be tested in the present selection method) and a sample to be used as a control are each labeled with different fluorescent substances, using two or more fluorescent substances, from the viewpoint of enabling simultaneous detection. Here, labeling of the samples is carried out by labeling mRNA in the samples, cDNA derived from the mRNA, or nucleic acids produced by transcription or amplification from cDNA.

Next, the hybridization is carried out between the above-mentioned labeled cDNA and the array to which a nucleic acid corresponding to a suitable gene or its fragment is immobilized. The hybridization may be performed according to any known processes under conditions that are appropriate for the array and the labeled cDNA to be used. For instance, the hybridization can be performed under the conditions described in Molecular Cloning, A laboratory manual, 2nd ed., 9.52-9.55 (1989).

The hybridization between the nucleic acids derived from the samples and the array is carried out, under the above-mentioned hybridization conditions. When much time is needed for the time period required for procedures from the collection of samples to the determination of expression levels of genes, the degradation of mRNA may take place due to actions of ribonuclease. In order to determine the difference in the gene expressions in the samples to be tested (i.e., tumor cells or biopsies from oligodendroglial tumor patients) and the gene expressions in a control sample, it is preferable that the mRNA levels in both of these samples are adjusted using a standard gene with relatively little alterations in expressions.

Thereafter, by comparing the hybridization results of the samples to be tested with those of the control sample, genes exhibiting differential expression levels in both samples can be detected. Concretely, a signal which is appropriate depending upon the method of labeling used is detected for the array which is subjected to hybridization with the nucleic acid sample labeled by the method as described above, whereby the expression levels in the samples to be tested can be compared with the expression level in the control sample for each of the genes on the array.

The genes thus obtained which have a significant difference in signal intensities are genes each of which expression is altered specifically for certain oligodendroglial tumor classes.

The present invention also provides a computer-readable medium comprising a plurality of digitally-encoded expression profiles wherein each profile of the plurality has a plurality of values, each value representing the expression of a gene that is differentially-expressed in an oligodendroglial tumor class. The invention also provides for the storage and retrieval of a collection of data relating to oligodendroglial tumor specific gene expression data of the present invention, including sequences and expression levels in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor).

For use in diagnostic, research, and therapeutic applications suggested above, kits are also provided by the invention. In the diagnostic and research applications such kits may include any or all of the following: assay reagents, buffers, oligodendroglial tumor class-specific nucleic acids or antibodies, hybridization probes and/or primers, antisense polynucleotides, ribozymes, arrays, antibodies, Fab fragments, capture peptides etc. In addition, the kits may include instructional materials containing directions (i.e., protocols) for the practice of the methods of this invention. While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials. One such internet site may provide a database of oligodendroglial tumor reference expression profiles useful for performing similarity clustering of a newly determined subject expression profiles with a large set of reference profiles of oligodendroglial subjects comprised in said database. Preferably the database includes clinically relevant data such as patient prognosis, effects of methods of treatment and other characteristics relating to the oligodendroglial tumor patient.

The invention encompasses for instance kits comprising an array of the invention and a computer-readable medium having digitally-encoded reference profiles with values representing the expression of nucleic acid molecules detected by the arrays. These kits are useful for assigning a brain tumor patient subject to an oligodendroglial tumor class.

In a preferred embodiment a kit-of-parts according to the invention comprises an oligonucleotide microarray according to the invention and means for comparing a gene expression profile determined by using said microarray with a database of oligodendroglial reference expression profiles. The present invention also comprises kits of parts suitable for performing a method of the invention as well as the use of the various products of the invention, including databases, microarrays, oligonucleotide probes and classification schemes in diagnostic or prognostic methods of the invention.

The present invention discloses a number of genes that are differentially-expressed in oligodendroglial tumor classes. These differentially-expressed genes are shown in Tables 3-7. Many of the treatment sensitivity-associated transcripts (Table 3) are involved in transcriptional regulation, interaction with the extracellular matrix or affect cytoskeletal dynamics. For example genes involved in regulation of transcription include: i) PAX8, a member of the paired box gene family of transcription factors; ii) Sp110, a protein that can function as an activator of transcription; iii) RENT1, a protein involved in mRNA nuclear export and nonsense-mediated mRNA decay; and iv) TNFSF13, a member of the tumor necrosis factor ligand family that activate transcription via e.g. NF-κB. TNFSF13 transgenic mice develop lymphoid tumors (Planelles, L. et al., (2004) Cancer Cell 6:399-408). Transcripts involved in the cellular interaction with the extracellular matrix include: i) MAN1C1, an α-mannosidase involved in the maturation of N-linked glycans; ii) CHSY1, which synthesizes chondroitin sulfate, a widely expressed glycosaminoglycan and iii) LGALS9, a member of the tandem-repeat type galectins that bind beta-galactoside. LGALS9 is expressed at high levels in distant metastasis of breast cancer (for review see (Hirashima, M. et al., (2004) Glycoconj. J. 19:593-600). Also two treatment sensitivity associated transcripts that are involved in regulation of cytoskeletal dynamics were identified: i) ARPC1B, involved in the branching of actin filaments and downregulated in gastric cancers; and ii) IQGAP1, a scaffolding protein that interacts with components of the cytoskeleton. Overexpression of IQGAP1 enhances cell migration (Mataraza, J. M. et al., (2003) J. Biol. Chem. 278: 41237-41245). Other genes expressed at high levels in chemoresistant oligodendroglial tumors include i) AQP1, a water channel often highly expressed in malignant gliomas that plays a role in migration and neovascularization of tumors; ii) TRIM56, a member of the tripartite motif family and iii) ARH, an adaptor protein that interacts with the LDL receptor. In summary, the genes identified in this invention that are associated with treatment sensitivity (Table 3) are involved in several discrete cellular processes and further study on these transcripts may help identify the molecular mechanisms that underlie treatment sensitivity.

Comparison of expression profiles to patient survival after diagnosis identified 103 differentially expressed probesets (Table 4). The observation that many genes are differentially expressed suggests that different molecular pathways are affected in the tumors of short and long survivors. The genetic background of the tumor therefore appears to be an important factor in determining the prognosis of the patient, although other factors also can contribute significantly to patient survival (e.g. tumor location). Therefore, genes that are differentially expressed between long and short survivors can help identify patient subgroups that are associated with favorable prognosis. Functional analysis reveals that many transcripts upregulated in short survivors are involved in the regulation of transcription. Examples include, i) BTEB1, a member of the SP1-like/KLF family of transcription regulators, ii) BCL10, an activator NF-κB, iii) DR1, a transcriptional repressor, iv) JUN, part of the AP1 transcription factor complex, v) PTPN12 and vi) PTP4A2, members of the protein tyrosine phosphatase family that regulate processes including cell growth, differentiation, mitotic cycle, and oncogenic transformation, vii) SFRS4, a member of the SR family of splicing factors, and viii) LMO4, a LIM domain containing protein that may play a role as a transcriptional regulator. In contrast, transcripts encoding proteins involved in RNA translation are downregulated in short survivors. They include five ribosomal proteins (RPL24, RPL3, PRL7, RPLP2 and RPS3) and proteins involved in post-transcriptional modification like CUGBP1 and RBM4.

This invention shows that expression profiling can identify transcripts associated with chromosomal aberrations, therapeutic response and survival after diagnosis in patients suffering from oligodendroglial tumors. As described above this knowledge can be used to identify patient classes with a high likelihood to respond to treatment and patient classes with favorable prognosis.

The following examples are offered by way of illustration and not by way of limitation.

### Example

### Methods

### Tumor Samples:

Patients were chosen with (anaplastic) oligodendroglioma or mixed oligoastrocytoma with enhancing disease at the time of chemotherapy. Patients were treated in a single institution (Erasmus MC) in clinical trials evaluating the efficacy of Temozolomide or PCV. Only patients with an evaluable for response to chemotherapy were included in this study. Treatment response was evaluated by MRI and scored according to McDonald's criteria (Macdonald D. R. et al., (1990) J. Clin. Oncol. 8:1277-1280). Tumor size was defined as the product of the two largest perpendicular tumor diameters. Complete response (CR) was defined as disappearance of all contrast-enhancing tumor on two subsequent scans at least one month apart, the patient being off steroids and neurologically stable or improved. Partial response (PR) was defined as ≧50% reduction in tumor area on two subsequent scans at least one-month apart, steroids stable or decreased and neurologically stable or improved. Progressive disease (PD) was defined as ≧25% increase in tumor area, new tumor on MRI or neurological deterioration and steroids stable or increased. All other situations were considered stable disease (SD). Samples were collected immediately after surgical resection, snap frozen, and stored at −800 C in the Erasmus MC brain tumor tissue bank. Samples were visually inspected on 10 μm H&E stained frozen sections by the neuropathologist (J.M.K). Samples with less than 80% tumor were omitted from this study. Tissue adjacent to the inspected sections was subsequently used for nucleic acid isolation. Using these criteria, 28 oligodendroglial tumors were selected (Table 1). Four additional tumor samples with insufficient RNA quantity for array analysis were selected for confirmation of differentially expressed genes using QPCR.

### Nucleic Acid Isolation:

Tissues were homogenized using a polytron following which RNA and genomic DNA were extracted using Trizol (Life-Technologies) according to the manufacturers instructions. Total RNA, present in the aqueous phase after extraction, was precipitated in isopropanol, redissolved in diethyl-pyrocarbonate treated water and further purified on RNeasy mini columns (Qiagen). Genomic DNA present in the organic phase was precipitated using ethanol, washed in 0.1M Na-citrate, 10% ethanol and dissolved in 8 mM NaOH whereafter the pH was adjusted to 8.4 using 1M Hepes (free acid).

cDNA Synthesis And Array Hybridization

RNA quality was assessed on agarose gel and Bioanalyser (Agilent). cDNA synthesis and cRNA labeling was performed using the alternative protocol for one-cycle cDNA synthesis. Biotin-labeled cRNA was generated using the ENZO Highyield RNA transcript labeling kit (ENZO life sciences inc, NY). Affymetrix (Santa Clara, Calif.) HG U133-plus2 microarrays were hybridized overnight with 15 μg biotin labeled cRNA. 54.675 probesets (a probeset is a set of oligonucleotide probes that examines the expression of a single transcript) are spotted on these arrays allowing expression profiling of virtually all human transcripts. Multiple probesets may be directed against the same transcript. Microarrays were then washed using fluidics stations according to standard Affymetrix protocols.

### Microsatellite Analysis

Microsatellites were amplified by PCR on 10 ng genomic DNA using forward and reversed primers and a fluorescently labeled M13 (−21) primer. Primers and cycling conditions are stated in supplementary table 1. PCR products were precipitated, dissolved in formamide and run on an ABI 3100 genetic analyzer (Applied Biosystems). Samples were analyzed using Genescan 3.7 software (Applied Biosystems) and scored by two independent researchers. Since non-neoplastic tissues were not available for most of the tumor samples, allelic losses were statistically determined as described (Harkes I. C., et al. (2003) Br. J. Cancer 89:2289-2292). Allelic loss was assumed when the tumor sample had a homozygous allele pattern for all microsatellites within the locus (P<0.05 for each locus).

### Fluorescence In Situ Hybridization

1p/19q status of samples with non-informative microsatellite analysis was determined using Fluorescence In Situ Hybridization (FISH) as previously described (Stege E. M. et al., (2005) Cancer 103:802-809). Locus-specific probes for 1p36 (D1S32), centromere 1 (pUC1.77), 19q13.4 (Bac clone 426G3), and 19p13 (Bac clones 957I1, 153P24, and 959O6) were labeled with either biotin-16-dUTP, digoxigenin-16-dUTP (Roche Diagnostics, Mannheim, Germany) or Spectrum Orange (Vysis Illinois, USA) as previously described (23). Probes were detected using FITC-labeled sheep-anti-digoxigenin (Roche Diagnostics) and/or CY3-labeled avidin (Brunschwig Chemie, Amsterdam, The Netherlands). Nuclei were counterstained with 4′,6-diamidino-2-phenylindole (DAPI). Sixty non-overlapping nuclei were enumerated per hybridization. Ratios were calculated as the number of signals of the marker divided by the number of signals of the reference. Ratio <0.80 were considered allelic loss.

### Semi-Quantitative RT-PCR

Semi-quantitative RT-PCR was performed using SYBR Green PCR master mix (Applied Biosystems) according to the manufacturers instructions. Expression levels were evaluated relative to HPRT and PDGB controls. Intron spanning primers were designed against 16 genes (supplementary table 2). All primers had an amplification efficiency >80% (determined by serial dilution) and generated a single amplification product at a temperature above 77° C. (determined by melting point analysis). Cycling was performed on an ABI7700 sequence detection system (Applied Biosystems); cycling conditions are stated in supplementary table 2. Amplification of the EFGR receptor was determined by semi-quantitative PCR using identical conditions as described above. 20 ng genomic DNA was used for each reaction. The amount of product amplified using genomic EGFR primers was compared to the amount of product amplified using primers on different chromosomes lying within the F3 and the FGFR3 loci. Statistical analysis was performed using the Mann Whitney U test (eatworms.swmed.edu/˜leon/stats/utest.cgi), values are ±SEM.

### Data Analysis:

Arrays were omitted from the analysis when the number of present calls <35% and when the 5′/3′ ratio of GAPDH controls >3. Probesets that were absent (according to Affymetrix MAS5.0 software) in at least 33 of the 34 microarrays were omitted from further analysis. Raw intensities of the remaining probesets (36875) of each chip were log 2 transformed and normalized using quantile normalization. For each probeset, the geometric mean of the hybridization intensities of all samples was calculated. The level of expression of each probeset was determined relative to this geometric mean and log 2 transformed. The geometric mean of the hybridization signal of all samples was used to ascribe equal weight to gene-expression levels. Unsupervised clustering was performed using Omniviz version 3.6.0 (Omniviz, Maynard, Mass.) software. Probesets whose expression levels differed more than 2 fold from the geometric mean in at least one sample were selected for the unsupervised clustering analysis. Similarities between samples is plotted using Omniviz software as Pearson's correlations.

Differentially expressed genes were identified using statistical analysis of microarrays (SAM analysis) (Tusher V. G. et al., Proc. Natl. Acad. Sci. U.S.A. 98:5116-5121). Such supervised analysis correlates gene expression with an external variable. SAM calculates a score for each probeset on the basis of the change in expression relative to the SD of all measurements. Unless otherwise indicated, analyses were performed using stringent statistical parameters with a false discovery rate (FDR) of less than 1 probeset. Differentially expressed probesets were imported into Spotfire DecisionSite (Spotfire, Somerville, Mass.) to perform principle components analysis (PCA) and hierarchical clustering. Data were log 2 transformed followed by calculation of the z-score for each probeset. PCA structures a dataset using as few variables as possible and is a mathematical way to reduce data dimensionality. PCA summarizes the most important variance in a dataset as principle components. For more information on the use of PCA in microarray analysis microarrays see (Raychaudhuri S. et al. (2000), In: Hunter L, Altman B, Dunker A K, Klein T E, Lauderdale K, editors. Pacific Symposium on Biocomputing 1999. Honolulu, Hi.: World Scientific Press; 2000) and references therein. Hierarchical clustering groups data based on their similarities in gene expression profiles. Weighted average was used to perform most clustering analysis, in which the distance between two clusters is defined as the average of distances between all pairs of objects. Unlike clustering based on unweighted averages, the weighted average ascribes equal weight to the two branches of the dendrogram that are about to be fused. Ward's hierarchical clustering method forms groups in a manner that minimizes the loss associated with each grouping. At each step in this analysis, the two clusters whose fusion results in minimum increase in information loss are combined.

### Results

### Samples:

Patient data, histological diagnosis, chromosomal aberrations, and response to chemotherapy are summarized in table 1. In total we performed expression analysis on 28 oligodendroglial tumors (2 lowgrade and 26 anaplastic oligodendrogliomas), and 6 control brain samples (4 samples from whole cortex, 2 from white matter only). We identified 14/28 samples (50%) with loss of most/all of the short arm of chromosome 1 (sample 18 had a predicted loss distal to 1p33) and 16/28 (57%) samples with loss of 19q (see Table 1). Most tumors showed combined loss or retention of 1p and 19q: only three tumors showed loss of 19q without loss of 1p, one showed LOH on 1p35.2 without loss of 19q. EGFR amplification and LOH on 10q was identified in 4/28 (14%) oligodendroglial tumors, three of which showed combined EGFR amplification and 10q LOH. When comparing the response rate (CR+PR vs. PD+SD) to loss of the telomeric end of chromosome 1, a response to chemotherapy was observed in 12/14 (86%) samples with 1p35.2 LOH and 6/14 (43%) without loss of 1p35.2. Similar results were obtained when comparing the response rate to LOH on 19q or to combined LOH on 1p and 19q (table 1). All four tumors in which the EGFR genomic region was amplified had retained both copies of 1p and 19q and showed no response to chemotherapy (progressive disease for all). 3/4 tumors with 10q LOH showed no response to treatment.

### Unsupervised Clustering:

Unsupervised clustering identifies a number of subgroups, summarized in FIG. 1. A first subgroup consists mainly of control samples but also includes low-grade tumor samples. Because the amount of tumor present in all samples was high (determined by visual inspection of sections prior to the sample used for expression profiling), this close homology to control brain tissue is likely to reflect an intrinsic property of low-grade oligodendroglial tumors. The low-grade oligodendroglioma samples have a higher homology to samples from whole cortex than to samples from white matter. Group II consists of tumor samples that have LOH on 1p and 19q and has a relatively good prognosis: All but one sample respond favorably to chemotherapy and most (4/6) patients with CR are found in this group. Patients in this group also have a relatively long survival both after diagnosis (15.3±3.6 years) and after surgical resection of the tumor (4.8±1.5 years). Group III has the worst prognosis: None of the tumors respond to chemotherapy, the average time of survival after diagnosis was short (1.9±0.2 years) as was the average time after surgical resection (1.5±0.3 years). All tumors of this subgroup have retained both copies of 1p and 19q and are characterized by an amplification of the EGFR locus. The samples between groups II and III have a more mixed appearance, there is some degree of correlation with both groups I and group III. Many samples with PR and all samples with SD are found in this group. Survival after diagnosis and surgical resection is intermediate between groups II and III: 8.3±1.5 and 2.3±0.3 years respectively.

Supervised Clustering: Tumor Vs. Controls

We first performed supervised clustering to identify genes that are differentially expressed between control and tumors tissue. SAM analysis identified 1881 differentially expressed probesets (˜1413 genes). Strongest downregulated transcripts in oligodendroglial tumors include those that encode proteins expressed in mature oligodendrocytes: myelin associated oligodendrocyte basic protein (MOBP), myelin oligodendrocyte glycoprotein (MOG), myelin associated glycoprotein (LAG), claudin 11 (CLDN11) and myelin basic protein (MBP). These transcripts are expressed (±SD) at 0.052±0.021 (4 probesets), 0.10±0.013 (4 probesets), 0.086 (1 probeset), 0.30±0.25 (2 probesets), and 0.21±0.17 (7 probesets) levels of control brain mRNA respectively. This downregulation was observed in each sample. The strong downregulation in low-grade samples confirms the hypothesis that their homology to control brain tissue (see FIG. 1) is a result of the genes expressed by the tumor. The downregulation of MOG was confirmed using RT-PCR (table 2).

It has been reported that PDGFRα is often highly expressed in oligodendroglial tumors (Riemenschneider M. J. et al., (2004) Acta Neuropathol. (Berlin) 107:277-282). However, this gene was not present in the set of tumor-associated genes identified by our screen. Closer inspection reveals that, although PDGFRα is on average upregulated 4.1 fold, the high variation of upregulation (4.1±4.7) indicates that this transcript is not a reliable marker for the amount of tumor present in the sample. In fact, we failed to observe any upregulation in 10/28 samples. The select upregulation of PDGFRα in a subset of samples was confirmed using RTPCR.

### Supervised Clustering on Chromosomal Aberrations

Supervised clustering was performed to identify genes associated with specific chromosomal losses. For this we compared expression profiles of samples with i) 1p LOH (n=9) vs. no loss (n=9), ii) 19q LOH (n=11) vs. no loss (n=7), and iii) combined 1p and 19q LOH (n=6) with no loss on either arm (n=6). SAM analysis identified 376, 64 and 60 probesets as being differentially expressed following loss of 1p, 19q or 1p and 19q respectively. Probesets are listed in supplementary table 3. Interestingly, many of the identified probesets are located on the lost chromosomal arm(s): 136/376 (36.1%) probesets are located on 1p, 25/64 (39.1%) on 19q and 49/60 (82%) on 1p or 19q. Of the differentially expressed genes located on the lost chromosomal arm(s), the ratio (═SD) loss vs. no loss is 0.53±0.22 (1p), 0.54±0.07 (19q) and 0.53±0.09 (1p and 19q) indicating that loss of one allele reduces expression levels by ˜50%. In fact, all but two of the differentially expressed probesets that are located on the lost chromosomal(s) are downregulated. This correlation between chromosomal loss and expression level therefore suggest that these genes have an allele-number dependent expression level. Furthermore, the differentially expressed genes can be identified across the entire chromosomal arms and suggests the entire arms have been lost.

Principle components analysis (PCA) and hierarchical clustering of genes associated with LOH on 1p and 19q is depicted in FIG. 2. All anaplastic oligodendrogliomas with combined loss/retention of 1p and 19q were correctly distributed by the first principal component axis, PCA1. This correct distribution includes 7 samples (2 samples that have retained both 1p and 19q copies and 5 samples with LOH on 1p and 19q) that were omitted from the clustering analysis. Further confirmation of a subset of differentially expressed genes by RT-PCR is shown in table 2 (including 4 additional oligodendroglial tumors).

Genes Associated with Chemosensitivity

We next performed supervised clustering to identify genes that are associated with response to chemotherapy. For this analysis we compared mRNA expression levels between tumors that show a response to chemotherapy (CR+PR), and those that do not (SD+PD). Such comparison using SAM (FDR<1 gene) identified 16 differentially expressed probesets that are listed in the supplementary table 3. 160 differentially expressed probesets (137 genes) were identified using less stringent statistical analysis (FDR=4.9%), of which 31 (27 genes) are located on chromosomes 1p or 19q (19%). Confirmation of differentially expressed genes was performed using RT-PCR on IQGAP, MAN1C1, TRIM56 and AQP1 transcripts (table 2).

PCA based on the 16 genes associated with chemotherapeutic response identifies three main subgroups (FIG. 3): Samples with no response to chemotherapy (SD and PD, red), samples with response to treatment (CR and PR, green), and control samples (gray). Similarly, hierarchical clustering also separates the majority of oligodendroglial tumors with response to chemotherapy from those that show no or little response to treatment (FIG. 3). Similar results were obtained when clustering was performed on 160 differentially expressed probesets identified using FDR=4.9%. Most oligodendroglial tumors were correctly distributed on their response to treatment by the first principal component axis, PCA1: PCA1>0 in 14/18 samples that respond to treatment whereas PCA1<0 in 10/10 samples with no response to treatment. Only 4/28 samples were therefore incorrectly classified based on expression of genes associated with chemosensitivity. In comparison, 8/28 samples are incorrectly classified when predicting response to treatment based on the 1p chromosomal status: 6/14 tumors without LOH on 1p show response to treatment and 2/14 with LOH on 1p do not respond to treatment.

Genes Associated with Survival

We next performed supervised clustering to identify genes associated with overall survival after diagnosis. For this analysis we compared expression profiles of tumors from patients with the shortest survival time (2.0±0.3 years, n=7) with those with the longest survival time (17.6±4.4 years, n=8) after diagnosis. SAM analysis identified 103 probesets (92 genes, see supplementary data) associated with patient survival. 30 (29%) of these probesets are located on either 1p or 19q chromosomal arms. PCA of survival-associated genes identifies three main clusters of samples: oligodendroglial tumors with short survival, oligodendroglial tumors with long survival and control samples. Low-grade samples cluster between control and tumor samples. Similar subgroups were identified by hierarchical clustering using these probesets (FIG. 4). It is interesting to note that the subgroups identified by hierarchical clustering are virtually identical to the subgroups that were identified by unsupervised clustering (FIG. 1). Most oligodendroglial tumors were correctly distributed on survival after diagnosis by the first principal component axis, PCA1: PCA1>0 in 12/14 samples with favorable prognosis (i.e. survival time >7 years after diagnosis) whereas PCA1<0 in 8/11 samples with relatively short survival after diagnosis (i.e. <7 years).

TABLE 1
Summary of patient data, histological diagnosis and response to
chemotherapy of samples used in this study.

| | | | sample | 1p | 19q | 10q | EGFR | | | | surv | |
|---|---|---|---|---|---|---|---|---|---|---|---|---|
| Sample | sex | age | type | status | status | status | ampl | Response | ther | surv tot | op | alive |
| 1 | F | 39 | control | | | | | | | | | no |
| 3 | F | | control | | | | | | | | | no |
| 4 | M | 63 | control | | | | | | | | | no |
| 7 | M | 63 | control | | | | | | | | | no |
| 8 | F | 45 | AOD | LOH | LOH | no | no | CR | PCV | 15 | 8.5 | yes |
| | | | | | | LOH | | | | | | |
| 9 | M | 35 | AOD | LOH | LOH | no | no | PR | temo | 13 | 2.7 | no |
| | | | | | | LOH | | | | | | |
| 10 | M | 59 | AOD | LOH | LOH | no | no | PR | temo | 9.8 | 1.5 | no |
| | | | | | | LOH | | | | | | |
| 11 | M | 44 | AOD | LOH | LOH | no | no | PR | PCV | 12 | 3.2 | no |
| | | | | | | LOH | | | | | | |
| 12 | F | 57 | AOD | no | LOH | no | no | PR | PCV | 19 | 1.9 | no |
| | | | | LOH | | LOH | | | | | | |
| 13 | M | 40 | AOD | LOH | LOH | no | no | PR | PCV | 24 | 1.9 | no |
| | | | | | | LOH | | | | | | |
| 14 | M | 59 | AOD | no | no | LOH | yes | PD | PCV | 2 | 1.6 | no |
| | | | | LOH | LOH | | | | | | | |
| 15 | F | 19 | AOD | no | no | no | no | CR | temo | 3.7 | 3.7 | no |
| | | | | LOH | LOH | LOH | | | | | | |
| 16 | M | 49 | AOA | no | LOH | no | no | SD | PCV | 10.9 | 1.2 | no |
| | | | | LOH | | LOH | | | | | | |
| 17 | M | 47 | AOD | no | no | no | no | PD | PCV | 4 | 3.9 | no |
| | | | | LOH | LOH | LOH | | | | | | |
| 18 | M | 34 | AOD | LOH | no | no | no | PR | PCV | 1.8 | 0.4 | no |
| | | | | | LOH | LOH | | | | | | |
| 20 | M | 50 | AOD | LOH | LOH | no | no | SD | temo | 11 | 1.3 | no |
| | | | | | | LOH | | | | | | |
| 21 | M | 32 | AOD | LOH | LOH | no | no | CR | PCV | 3.9 | 3.6 | yes |
| | | | | | | LOH | | | | | | |
| 22 | M | 55 | AOD | no | no | LOH | yes | PD | PCV | 1.5 | 1.4 | no |
| | | | | LOH | LOH | | | | | | | |
| 23 | F | 45 | AOD | LOH | LOH | no | no | PR | PCV | 19 | 6.1 | no |
| | | | | | | LOH | | | | | | |
| 24 | M | 43 | AOD | no | no | no | no | PR | PCV | 11 | 1.0 | no |
| | | | | LOH | LOH | LOH | | | | | | |
| 25 | M | 51 | AOD | LOH | LOH | no | no | PD | temo | 10 | 3.0 | no |
| | | | | | | LOH | | | | | | |
| 28 | M | 35 | AOD | LOH | LOH | no | no | CR | PCV | 2.2 | 2.2 | yes |
| | | | | | | LOH | | | | | | |
| 29 | M | 52 | AOD | no | no | LOH | yes | PD | temo | 2.3 | 2.1 | no |
| | | | | LOH | LOH | | | | | | | |
| 30 | M | 88 | control | | | | | | | | | no |
| 31 | F | 68 | control | | | | | | | | | no |
| 34 | M | 45 | AOD | no | no | no | yes | PD | PCV | 1.8 | 1.0 | no |
| | | | | LOH | LOH | LOH | | | | | | |
| 36 | F | 21 | AOA | no | LOH | no | no | PR | temo | 2.5 | 2.4 | no |
| | | | | LOH | | LOH | | | | | | |
| 37 | F | 33 | AOD | LOH | LOH | no | no | CR | PCV | 23 | 11.1 | no |
| | | | | | | LOH | | | | | | |
| 38 | F | 39 | OD | LOH | LOH | no | no | CR | PCV | 9.7 | 6.6 | no |
| | | | | | | LOH | | | | | | |
| 40 | M | 45 | AOD | LOH | LOH | no | no | PR | PCV | 16 | 3.5 | no |
| | | | | | | LOH | | | | | | |
| 41 | F | 39 | AOA | no | no | LOH | no | PR | PCV | 4.8 | 4.1 | no |
| | | | | LOH | LOH | | | | | | | |
| 42 | F | 37 | OD | no | no | no | no | PR | PCV | 8 | 8.0 | yes |
| | | | | LOH | LOH | LOH | | | | | | |
| 44 | M | 39 | AOD | no | no | no | no | SD | temo | 2.7 | 2.7 | no |
| | | | | LOH | LOH | LOH | | | | | | |
| 46 | M | 30 | AOD | no | no | no | no | SD | PCV | 6.3 | 2.5 | no |
| | | | | LOH | LOH | LOH | | | | | | |

Additional samples used for RT-PCR confirmation

| | | | | | | | | | | |
|---|---|---|---|---|---|---|---|---|---|---|
| 26 | M | 52 | OD | LOH | no | no | no | MR | PCV | yes |
| | | | | | loss | loss | | | | |
| 27 | M | 44 | AOD | no LOH | loss | no | no | stopped | PCV | yes |
| | | | | | | loss | | | | |
| 32 | M | 72 | control | | | | | | | no |
| 33 | M | 49 | AOD | LOH | loss | no | no | PR | PCV | yes |
| | | | | | | loss | | | | |
| 45 | | | AOD | no LOH | no | no | no | unknown | | |
| | | | | | loss | loss | | | | |

M: male;
F: female;
ctr: normal brain;
ctr/w: control brain white matter;
OD oligodendroglioma (grade II);
AOD: anaplastic oligodendroglioma,
AOA anaplastic oligoastrocytoma;
LOH: loss of heterozygosity;
ampl: amplification of the EGFR locus;
ther.: therapy: PCV: combination therapy of procarbazine, CCNU, and vincristine;
temo: temozolomide. Treatment response was scored according to McDonald's criteria (20) CR: complete response;
PR: partial response;
SD: stable disease;
PD: progressive disease. Surv tot: patient survival after diagnosis (years);
Surv op: patient survival after surgical resection of the sample used in this study.

TABLE 2
Confirmation of a subset of differentially expressed genes identified by
expression profiling. Differential expression of most transcripts was
reconfirmed by RT-PCR. The relative expression levels between control (either
no loss of 1p, 19q, no tumor or CR/PR) and test set (either LOH on 1p, 19q,
tumor or SD/PD) also remained similar on the array (rel expr array) and by
RT-PCR (rel expr QPCR).

| | marker | rel expr | rel expr | QPCR | QPCR | |
|---|---|---|---|---|---|---|
| gene | for | array | QPCR | ctr | marker | P |
| F3 | 1p LOH | 5.4 | 6.9 | 0.52 ± 0.21 | 3.63 ± 2.88 | p < 0.001 |
| IQGAP | 1p LOH | 2.8 | 4.6 | 0.48 ± 0.15 | 2.19 ± 1.09 | p < 0.001 |
| PPAP2B | 1p LOH | 3.3 | 4.4 | 2.38 ± 0.75 | 10.4 ± 8.1 | p < 0.005 |
| GNG12 | 1p LOH | 2.8 | 5.4 | 0.61 ± 0.23 | 3.28 ± 1.19 | p < 0.001 |
| MOG | tumor | 11.6 | 21.9 | 2.44 ± 0.84 | 53.4 ± 5.0 | p < 0.00001 |
| LANCL2 | EGFR | 9.6 | 15.7 | 4.84 ± 0.56 | 76.1 ± 25.6 | p < 0.005 |
| | ampl | | | | | |
| EGFR | EGFR | 6.3 | 14.4 | 33.3 ± 4.6 | 480 ± 112 | p < 0.005 |
| | ampl | | | | | |
| CASP3 | 19q | 2.0 | 1.7 | 2.57 ± 0.83 | 4.47 ± 1.41 | ns |
| | LOH | | | | | |
| ZNF222 | 19q | 2.4 | 1.6 | 0.34 ± 0.10 | 0.54 ± 0.17 | ns |
| | LOH | | | | | |
| DCDT | 19q | 4.0 | 4.8 | 0.65 ± 0.29 | 3.14 ± 0.79 | p < 0.005 |
| | LOH | | | | | |
| MAN1C1 | response | 3.5 | 3.0 | 2.09 ± 0.50 | 6.24 ± 1.51 | p < 0.05 |
| IQGAP1 | response | 2.4 | 2.3 | 0.99 ± 0.41 | 2.32 ± 1.28 | p < 0.05 |
| TRIM56 | response | 2.3 | 2.7 | 0.17 ± 0.05 | 0.47 ± 0.21 | p < 0.05 |
| AQP1 | response | 9.7 | 7.5 | 2.42 ± 1.21 | 18.2 ± 13.2 | p < 0.02 |

QPCR ctr: expression of the examined transcript in control samples (either no loss of 1p, 19q, no tumor or CR/PR) relative to PDGB expression levels;
QPCR marker: expression of the examined transcript in test samples (either LOH on 1p, 19q, no tumor or CR/PR) relative to PDGB expression levels. Statistical analysis was performed on QPCR ctr vs. marker using the Mann Whitney U test (two tailed), values are ±SE.

TABLE 3
Differentially expressed probesets, which are able to discriminate on
basis of response tot treatment

| Probe Set ID | Title | Gene Symbol | Location |
|---|---|---|---|
| 1552506_at | hypothetical protein | FLJ38464 | Chr: 9q34.11 |
| | FLJ38464 | | |
| 1554830_a_at | dudulin 2 | TSAP6 | Chr: 2q14.2 |
| 1555600_s_at | apolipoprotein L, 4 | APOL4 | Chr: 22q11.2-q13.2 |
| 1555852_at | transporter 1, ATP- | TAP1 | Chr: 6p21.3 |
| | binding cassette, sub- | | |
| | family B (MDR/TAP) | | |
| 1555997_s_at | insulin-like growth factor | IGFBP5 | Chr: 2q33-q36 |
| | binding protein 5 | | |
| 1556643_at | hypothetical protein | LOC93343 | Chr: 19p13.12 |
| | BC011840 | | |
| 1567628_at | CD74 antigen (invariant | CD74 | Chr: 5q32 |
| | polypeptide of major | | |
| | histocompatibility | | |
| | complex, class II | | |
| | antigen-associated) | | |
| 1568619_s_at | hypothetical protein | LOC162073 | Chr: 16p13.11 |
| | LOC162073 | | |
| 200660_at | S100 calcium binding | S100A11 | Chr: 1q21 |
| | protein A11 (calgizzarin) | | |
| 200673_at | lysosomal-associated | LAPTM4A | Chr: 2p24.3 |
| | protein transmembrane 4 | | |
| | alpha | | |
| 200791_s_at | IQ motif containing | IQGAP1 | Chr: 15q26.1 |
| | GTPase activating | | |
| | protein 1 | | |
| 200867_at | zinc finger protein 313 | ZNF313 | Chr: 20q13.13 |
| 200887_s_at | signal transducer and | STAT1 | Chr: 2q32.2 |
| | activator of transcription | | |
| | 1, 91 kDa | | |
| 201053_s_at | proteasome (prosome, | PSMF1 | Chr: 20p13 |
| | macropain) inhibitor | | |
| | subunit 1 (PI31) | | |
| 201125_s_at | integrin, beta 5 | ITGB5 | Chr: 3q21.2 |
| 201136_at | proteolipid protein 2 | PLP2 | Chr: Xp11.23 |
| | (colonic epithelium- | | |
| | enriched) | | |
| 201259_s_at | synaptophysin-like | SYPL | Chr: 7q22.2 |
| | protein | | |
| 201319_at | myosin regulatory light | MRCL3 | Chr: 18p11.31 |
| | chain MRCL3 | | |
| 201324_at | epithelial membrane | EMP1 | Chr: 12p12.3 |
| | protein 1 | | |
| 201325_s_at | epithelial membrane | EMP1 | Chr: 12p12.3 |
| | protein 1 | | |
| 201336_at | vesicle-associated | VAMP3 | Chr: 1p36.23 |
| | membrane protein 3 | | |
| | (cellubrevin) | | |
| 201339_s_at | sterol carrier protein 2 | SCP2 | Chr: 1p32 |
| 201464_x_at | v-jun sarcoma virus 17 | JUN | Chr: 1p32-p31 |
| | oncogene homolog | | |
| | (avian) | | |
| 201465_s_at | v-jun sarcoma virus 17 | JUN | Chr: 1p32-p31 |
| | oncogene homolog | | |
| | (avian) | | |
| 201531_at | zinc finger protein 36, | ZFP36 | Chr: 19q13.1 |
| | C3H type, homolog | | |
| | (mouse) | | |
| 201560_at | chloride intracellular | CLIC4 | Chr: 1p36.11 |
| | channel 4 | | |
| 201590_x_at | annexin A2 | ANXA2 | Chr: 15q21-q22 |
| 201817_at | ubiquitin-protein | KIAA0010 | Chr: 7q36.3 |
| | isopeptide ligase (E3) | | |
| 201887_at | interleukin 13 receptor, | IL13RA1 | Chr: Xq24 |
| | alpha 1 | | |
| 201954_at | actin related protein 2/3 | ARPC1B | Chr: 7q22.1 |
| | complex, subunit 1B, | | |
| | 41 kDa | | |
| 201963_at | fatty-acid-Coenzyme A | FACL2 | Chr: 4q34-q35 |
| | ligase, long-chain 2 | | |
| 202096_s_at | benzodiazapine receptor | BZRP | Chr: 22q13.31 |
| | (peripheral) | | |
| 202132_at | transcriptional co- | TAZ | Chr: 3q23-q24 |
| | activator with PDZ- | | |
| | binding motif (TAZ) | | |
| 202133_at | transcriptional co- | TAZ | Chr: 3q23-q24 |
| | activator with PDZ- | | |
| | binding motif (TAZ) | | |
| 202193_at | LIM domain kinase 2 | LIMK2 | Chr: 22q12.2 |
| 202377_at | leptin receptor | LEPR | Chr: 1p31 |
| 202803_s_at | integrin, beta 2 (antigen | ITGB2 | Chr: 21q22.3 |
| | CD18 (p95), lymphocyte | | |
| | function-associated | | |
| | antigen 1; macrophage | | |
| | antigen 1 (mac-1) beta | | |
| | subunit) | | |
| 202863_at | nuclear antigen Sp100 | SP100 | Chr: 2q37.1 |
| 203044_at | carbohydrate | CHSY1 | Chr: 15q26.3 |
| | (chondroitin) synthase 1 | | |
| 203132_at | retinoblastoma 1 | RB1 | Chr: 13q14.2 |
| | (including osteosarcoma) | | |
| 203153_at | interferon-induced | IFIT1 | Chr: 10q25-q26 |
| | protein with | | |
| | tetratricopeptide repeats 1 | | |
| 203236_s_at | lectin, galactoside- | LGALS9 | Chr: 17q11.2 |
| | binding, soluble, 9 | | |
| | (galectin 9) | | |
| 203275_at | interferon regulatory | IRF2 | Chr: 4q34.1-q35.1 |
| | factor 2 | | |
| 203379_at | ribosomal protein S6 | RPS6KA1 | Chr: 3 |
| | kinase, 90 kDa, | | |
| | polypeptide 1 | | |
| 203426_s_at | insulin-like growth factor | IGFBP5 | Chr: 2q33-q36 |
| | binding protein 5 | | |
| 203567_s_at | tripartite motif-containing | TRIM38 | Chr: 6p21.3 |
| | 38 | | |
| 203735_x_at | Homo sapiens | — | — |
| | transcribed sequence | | |
| | with weak similarity to | | |
| | protein ref: NP_060312.1 | | |
| | (H. sapiens) hypothetical | | |
| | protein FLJ20489 [Homo | | |
| | sapiens] | | |
| 203879_at | phosphoinositide-3- | PIK3CD | Chr: 1p36.2 |
| | kinase, catalytic, delta | | |
| | polypeptide | | |
| 203973_s_at | KIAA0146 protein | KIAA0146 | Chr: 8q11.21 |
| 204017_at | KDEL (Lys-Asp-Glu-Leu) | KDELR3 | Chr: 22q13.1 |
| | endoplasmic reticulum | | |
| | protein retention receptor 3 | | |
| 206515_at | cytochrome P450, family | CYP4F3 | Chr: 19p13.2 |
| | 4, subfamily F, | | |
| | polypeptide 3 | | |
| 207542_s_at | aquaporin 1 (channel- | AQP1 | Chr: 7p14 |
| | forming integral protein, | | |
| | 28 kDa) | | |
| 207753_at | zinc finger protein 304 | ZNF304 | Chr: 19q13.4 |
| 208540_x_at | — | — | — |
| 208789_at | polymerase I and | PTRF | Chr: 17q21.31 |
| | transcript release factor | | |
| 208966_x_at | interferon, gamma- | IFI16 | Chr: 1q22 |
| | inducible protein 16 | | |
| 209047_at | aquaporin 1 (channel- | AQP1 | Chr: 7p14 |
| | forming integral protein, | | |
| | 28 kDa) | | |
| 209091_s_at | SH3-domain GRB2-like | SH3GLB1 | Chr: 1p22 |
| | endophilin B1 | | |
| 209619_at | CD74 antigen (invariant | CD74 | Chr: 5q32 |
| | polypeptide of major | | |
| | histocompatibility | | |
| | complex, class II | | |
| | antigen-associated) | | |
| 209762_x_at | SP110 nuclear body | SP110 | Chr: 2q37.1 |
| | protein | | |
| 209823_x_at | major histocompatibility | HLA-DQB1 | Chr: 6p21.3 |
| | complex, class II, DQ | | |
| | beta 1 | | |
| 209949_at | neutrophil cytosolic | NCF2 | Chr: 1q25 |
| | factor 2 (65 kDa, chronic | | |
| | granulomatous disease, | | |
| | autosomal 2) | | |
| 209969_s_at | signal transducer and | STAT1 | Chr: 2q32.2 |
| | activator of transcription | | |
| | 1, 91 kDa | | |
| 210426_x_at | RAR-related orphan | RORA | Chr: 15q21-q22 |
| | receptor A | | |
| 210427_x_at | annexin A2 | ANXA2 | Chr: 15q21-q22 |
| 210582_s_at | LIM domain kinase 2 | LIMK2 | Chr: 22q12.2 |
| 210829_s_at | single-stranded DNA | SSBP2 | Chr: 5q14.1 |
| | binding protein 2 | | |
| 210840_s_at | IQ motif containing | IQGAP1 | Chr: 15q26.1 |
| | GTPase activating | | |
| | protein 1 | | |
| 211168_s_at | regulator of nonsense | RENT1 | Chr: 19p13.2-p13.11 |
| | transcripts 1 | | |
| 211366_x_at | caspase 1, apoptosis- | CASP1 | Chr: 11q23 |
| | related cysteine protease | | |
| | (interleukin 1, beta, | | |
| | convertase) | | |
| 211429_s_at | serine (or cysteine) | SERPINA1 | Chr: 14q32.1 |
| | proteinase inhibitor, | | |
| | clade A (alpha-1 | | |
| | antiproteinase, | | |
| | antitrypsin), member 1 | | |
| 211495_x_at | tumor necrosis factor | TNFSF13 | Chr: 17p13.1 |
| | (ligand) superfamily, | | |
| | member 13 | | |
| 211561_x_at | mitogen-activated protein | MAPK14 | Chr: 6p21.3-p21.2 |
| | kinase 14 | | |
| 211612_s_at | interleukin 13 receptor, | IL13RA1 | Chr: Xq24 |
| | alpha 1 | | |
| 211656_x_at | major histocompatibility | HLA-DQB1 | Chr: 6p21.3 |
| | complex, class II, DQ | | |
| | beta 1 | | |
| 211733_x_at | sterol carrier protein 2 | SCP2 | Chr: 1p32 |
| 211749_s_at | vesicle-associated | VAMP3 | Chr: 1p36.23 |
| | membrane protein 3 | | |
| | (cellubrevin) | | |
| 211924_s_at | plasminogen activator, | PLAUR | Chr: 19q13 |
| | urokinase receptor | | |
| 211959_at | insulin-like growth factor | IGFBP5 | Chr: 2q33-q36 |
| | binding protein 5 | | |
| 212203_x_at | interferon induced | IFITM3 | Chr: 11p15.5 |
| | transmembrane protein 3 | | |
| | (1-8U) | | |
| 212268_at | serine (or cysteine) | SERPINB1 | Chr: 6p25 |
| | proteinase inhibitor, | | |
| | clade B (ovalbumin), | | |
| | member 1 | | |
| 212687_at | LIM and senescent cell | LIMS1 | Chr: 2q12.3 |
| | antigen-like domains 1 | | |
| 212859_x_at | metallothionein 1E | MT1E | Chr: 16q13 |
| | (functional) | | |
| 213293_s_at | tripartite motif-containing | TRIM22 | Chr: 11p15 |
| | 22 | | |
| 213446_s_at | IQ motif containing | IQGAP1 | Chr: 15q26.1 |
| | GTPase activating | | |
| | protein 1 | | |
| 213503_x_at | annexin A2 | ANXA2 | Chr: 15q21-q22 |
| 213504_at | COP9 subunit 6 (MOV34 | COPS6 | Chr: 7q22.1 |
| | homolog, 34 kD) | | |
| 213698_at | zinc finger protein 258 | ZNF258 | Chr: 1p34.2 |
| 214087_s_at | myosin binding protein | MYBPC1 | Chr: 12q23.3 |
| | C, slow type | | |
| 214180_at | Homo sapiens | — | — |
| | transcribed sequence | | |
| | with weak similarity to | | |
| | protein ref: NP_060265.1 | | |
| | (H. sapiens) hypothetical | | |
| | protein FLJ20378 [Homo | | |
| | sapiens] | | |
| 214257_s_at | hypothetical protein | FLJ21272 | Chr: 1q21.2 |
| | FLJ21272 | | |
| 214684_at | MADS box transcription | MEF2A | Chr: 15q26 |
| | enhancer factor 2, | | |
| | polypeptide A (myocyte | | |
| | enhancer factor 2A) | | |
| 214791_at | hypothetical protein | LOC93349 | Chr: 2q37.1 |
| | BC004921 | | |
| 216526_x_at | major histocompatibility | HLA-C | Chr: 6p21.3 |
| | complex, class I, C | | |
| 216598_s_at | chemokine (C-C motif) | CCL2 | Chr: 17q11.2-q21.1 |
| | ligand 2 | | |
| 217388_s_at | kynureninase (L- | KYNU | Chr: 2q22.3 |
| | kynurenine hydrolase) | | |
| 217730_at | PP1201 protein | PP1201 | Chr: 2p24.3-p24.1 |
| 217746_s_at | programmed cell death 6 | PDCD6IP | Chr: 3p22.3 |
| | interacting protein | | |
| 217788_s_at | UDP-N-acetyl-alpha-D- | GALNT2 | Chr: 1q41-q42 |
| | galactosamine:polypeptide | | |
| | N- | | |
| | acetylgalactosaminyltransferase | | |
| | 2 (GalNAc-T2) | | |
| 218154_at | hypothetical protein | FLJ12150 | Chr: 8q24.3 |
| | FLJ12150 | | |
| 218162_at | HNOEL-iso protein | HNOEL-iso | Chr: 1p13.1 |
| 218247_s_at | hypothetical protein | LOC51320 | Chr: 18q21.1 |
| | LOC51320 | | |
| 218418_s_at | KIAA1518 protein | KIAA1518 | Chr: 19p13.2 |
| 218673_s_at | ubiquitin activating | GSA7 | Chr: 3p25.2 |
| | enzyme E1-like protein | | |
| 218802_at | hypothetical protein | FLJ20647 | Chr: 4q25 |
| | FLJ20647 | | |
| 218918_at | mannosidase, alpha, | MAN1C1 | Chr: 1p35 |
| | class 1C, member 1 | | |
| 218943_s_at | DEAD/H (Asp-Glu-Ala- | RIG-I | Chr: 9p12 |
| | Asp/His) box polypeptide | | |
| 219505_at | cat eye syndrome | CECR1 | Chr: 22q11.2 |
| | chromosome region, | | |
| | candidate 1 | | |
| 219706_at | chromosome 20 open | C20orf29 | Chr: 20p13 |
| | reading frame 29 | | |
| 219751_at | hypothetical protein | FLJ21148 | Chr: 16q13 |
| | FLJ21148 | | |
| 220088_at | complement component | C5R1 | Chr: 19q13.3-q13.4 |
| | 5 receptor 1 (C5a ligand) | | |
| 220407_s_at | transforming growth | TGFB2 | Chr: 1q41 |
| | factor, beta 2 | | |
| 220477_s_at | chromosome 20 open | C20orf30 | Chr: 20p13 |
| | reading frame 30 | | |
| 221773_at | ELK3, ETS-domain | ELK3 | Chr: 12q23 |
| | protein (SRF accessory | | |
| | protein 2) | | |
| 221790_s_at | LDL receptor adaptor | ARH | Chr: 1p36-p35 |
| | protein | | |
| 222448_s_at | UMP-CMP kinase | UMP-CMPK | — |
| 223047_at | chemokine-like factor | CKLFSF6 | Chr: 3p22.3 |
| | super family 6 | | |
| 223165_s_at | inositol hexaphosphate | IHPK2 | Chr: 3p21.31 |
| | kinase 2 | | |
| 223376_s_at | brain protein I3 | BRI3 | Chr: 7q22.1 |
| 223642_at | Zic family member 2 | ZIC2 | Chr: 13q32 |
| | (odd-paired homolog, | | |
| | Drosophila) | | |
| 223681_s_at | InaD-like protein | INADL | Chr: 1p32.1 |
| 224584_at | chromosome 20 open | C20orf30 | Chr: 20p13 |
| | reading frame 30 | | |
| 224840_at | FK506 binding protein 5 | FKBP5 | Chr: 6p21.3-21.2 |
| 224856_at | FK506 binding protein 5 | FKBP5 | Chr: 6p21.3-21.2 |
| 225267_at | karyopherin alpha 4 | KPNA4 | Chr: 3q25.33 |
| | (importin alpha 3) | | |
| 225415_at | rhysin 2 | LOC151636 | Chr: 3q21.1 |
| 225869_s_at | unc-93 homolog B1 (C. elegans) | UNC93B1 | Chr: 11q13 |
| 226040_at | Homo sapiens cDNA | — | — |
| | FLJ11958 fis, clone | | |
| | HEMBB1000996. | | |
| 226074_at | hypothetical protein | FLJ32332 | Chr: 3p21.31 |
| | FLJ32332 | | |
| 226621_at | fibrinogen, gamma | FGG | Chr: 4q28 |
| | polypeptide | | |
| 226628_at | THO complex 2 | THOC2 | Chr: Xq25-q26.3 |
| 226694_at | A kinase (PRKA) anchor | AKAP2 | Chr: 9q31-q33 |
| | protein 2 | | |
| 227013_at | LATS, large tumor | LATS2 | Chr: 13q11-q12 |
| | suppressor, homolog 2 | | |
| | (Drosophila) | | |
| 227066_at | similar to MOB-LAK | LOC148932 | Chr: 1p34.1 |
| 227474_at | paired box gene 8 | PAX8 | Chr: 2q12-q14 |
| 227792_at | Homo sapiens cDNA: | — | — |
| | FLJ22994 fis, clone | | |
| | KAT11918. | | |
| 227801_at | tumor suppressor TSBF1 | TSBF1 | Chr: 3q26.1 |
| 227837_at | hypothetical protein | FLJ20309 | Chr: 2q33.3 |
| | FLJ20309 | | |
| 227882_at | fukutin-related protein | FKRP | Chr: 19q13.33 |
| 228042_at | ADP-ribosylarginine | ADPRH | Chr: 3q13.31-q13.33 |
| | hydrolase | | |
| 228229_at | KIAA1951 protein | KIAA1951 | Chr: 19q13.31 |
| 228369_at | trinucleotide repeat | TNRC5 | Chr: 6pter-p12.1 |
| | containing 5 | | |
| 228410_at | GRB2-associated | GAB3 | Chr: Xq28 |
| | binding protein 3 | | |
| 228425_at | Homo sapiens, clone | — | — |
| | IMAGE: 4820851, mRNA | | |
| 228651_at | hypothetical gene | | Chr: 1 |
| | supported by AK075366 | | |
| 228949_at | putative NFkB activating | FLJ23091 | Chr: 1p31.2 |
| | protein 373 | | |
| 228980_at | hypothetical gene | | Chr: 17q21.1 |
| | supported by AK091492; | | |
| | AL831912 | | |
| 229101_at | hypothetical protein | LOC150166 | Chr: 22q11.21 |
| | LOC150166 | | |
| 229143_at | CCR4-NOT transcription | CNOT3 | Chr: 19q13.4 |
| | complex, subunit 3 | | |
| 229812_at | ubiquitin specific | USP31 | Chr: 1p36.12 |
| | protease 31 | | |
| 230636_s_at | basic transcription | BTEB1 | Chr: 9q13 |
| | element binding protein 1 | | |
| 231876_at | tripartite motif-containing | TRIM56 | Chr: 7q22.1 |
| | 56 | | |
| 233103_at | Homo sapiens cDNA | — | — |
| | FLJ14109 fis, clone | | |
| | MAMMA1001322, | | |
| | moderately similar to B- | | |
| | CELL GROWTH | | |
| | FACTOR PRECURSOR. | | |
| 240277_at | solute carrier family 30 | SLC30A7 | Chr: 1p21.2 |
| | (zinc transporter), | | |
| | member 7 | | |
| 240656_at | Homo sapiens | — | — |
| | transcribed sequences | | |
| 242521_at | Homo sapiens, similar to | — | — |
| | Alu subfamily SQ | | |
| | sequence contamination | | |
| | warning entry, clone | | |
| | IMAGE: 4342162, mRNA | | |
| 40524_at | protein tyrosine | PTPN21 | Chr: 14q31.3 |
| | phosphatase, non- | | |
| | receptor type 21 | | |
| 57163_at | elongation of very long | ELOVL1 | Chr: 1p34.1 |
| | chain fatty acids | | |
| | (FEN1/Elo2, SUR4/Elo3, | | |
| | yeast)-like 1 | | |
| AFFX- | — | — | — |
| HUMISGF3A/M97935_3_at | | | |
| AFFX- | — | — | — |
| HUMISGF3A/M97935_MB_at | | | |

TABLE 4
Differentially expressed probesets, which are able to discriminate on
basis of patient survival

| | | | | ratio |
|---|---|---|---|---|
| Probe Set ID | Title | Gene Symbol | Location | short/long |
| 200902_at | 15 kDa selenoprotein | SEP15 | Chr: 1p31 | 1.92 |
| 231057_at | Myotubularin related protein 2 | MTMR2 | Chr: 11q21 | 0.38 |
| 232929_at | Homo sapiens cDNA FLJ13240 | — | Chr: 3q13.31 | 0.40 |
| | fis, clone OVARC1000496. | | | |
| 213156_at | Homo sapiens, clone | IMAGE: 4214654 | Chr: 3q13.31 | 0.44 |
| | IMAGE: 4214654, mRNA | | | |
| 227082_at | Homo sapiens mRNA; cDNA | — | Chr: 3q13.31 | 0.39 |
| | DKFZp586K1922 (from clone | | | |
| | DKFZp586K1922) | | | |
| 227121_at | Homo sapiens mRNA; cDNA | — | Chr: 3q13.31 | 0.43 |
| | DKFZp586K1922 (from clone | | | |
| | DKFZp586K1922) | | | |
| 239545_at | O-acetyltransferase | CAS1 | Chr: 7q21.3 | 0.47 |
| 229624_at | similar to OPA3 protein; Optic | LOC401922 | Chr: 19q13.32 | 1.96 |
| | atrophy 3 (Iraqi-Jewish optic | | | |
| | atrophy plus) | | | |
| 235384_at | similar to RP2 protein, | LOC390916 | Chr: 19q13.11 | 2.37 |
| | testosterone-regulated - ricefield | | | |
| | mouse (Mus caroli) | | | |
| 229075_at | Homo sapiens transcribed | — | Chr: 4q28.1 | 1.61 |
| | sequences | | | |
| 237803_x_at | Homo sapiens transcribed | — | | 0.34 |
| | sequences | | | |
| 241435_at | V-ets erythroblastosis virus E26 | ETS1 | Chr: 11q23.3 | 0.36 |
| | oncogene homolog 1 (avian) | | | |
| 240216_at | CDNA FLJ25794 fis, clone | — | Chr: 3q13.31 | 0.42 |
| | TST07014 | | | |
| 239577_at | Homo sapiens, clone | — | — | 0.42 |
| | IMAGE: 4182817, mRNA | | | |
| 226189_at | Homo sapiens, clone | IMAGE: 4794726 | Chr: 7p21.1 | 2.38 |
| | IMAGE: 4794726, mRNA | | | |
| 218694_at | ALEX1 protein | ALEX1 | Chr: Xq21.33-q22.2 | 0.66 |
| 226291_at | amyotrophic lateral sclerosis 2 | ALS2 | Chr: 2q33.2 | 0.78 |
| | (juvenile) | | | |
| 223251_s_at | ankyrin repeat domain 10 | ANKRD10 | Chr: 13q34 | 0.42 |
| 224810_s_at | ankyrin repeat domain 13 | ANKRD13 | Chr: 12q24.12 | 0.65 |
| 200782_at | annexin A5 | ANXA5 | Chr: 4q28-q32 | 2.92 |
| 205711_x_at | ATP synthase, H+ transporting, | ATP5C1 | Chr: 10q22-q23 | 0.69 |
| | mitochondrial F1 complex, | | | |
| | gamma polypeptide 1 | | | |
| 208870_x_at | ATP synthase, H+ transporting, | ATP5C1 | Chr: 10q22-q23 | 0.68 |
| | mitochondrial F1 complex, | | | |
| | gamma polypetide 1 | | | |
| 205263_at | B-cell CLL/lymphoma 10 | BCL10 | Chr: 1p22 | 1.75 |
| 203543_s_at | basic transcription element | BTEB1 | Chr: 9q13 | 2.66 |
| | binding protein 1 | | | |
| 217928_s_at | chromosome 11 open reading | C11orf23 | Chr: 11q13 | 0.57 |
| | frame 23 | | | |
| 218796_at | chromosome 20 open reading | C20orf42 | Chr: 20p12.3 | 0.09 |
| | frame 42 | | | |
| 217752_s_at | cytosolic nonspecific dipeptidase | CN2 | Chr: 18q22.3 | 1.59 |
| | (EC 3.4.13.18) | | | |
| 222409_at | coronin, actin binding protein, 1C | CORO1C | Chr: 12q24.1 | 0.52 |
| 204264_at | carnitine palmitoyltransferase II | CPT2 | Chr: 1p32 | 1.59 |
| 209489_at | CUG triplet repeat, RNA binding | CUGBP1 | Chr: 11p11 | 0.67 |
| | protein 1 | | | |
| 225434_at | death effector domain-containing | DEDD2 | Chr: 19q13.31 | 2.33 |
| | DNA binding protein 2 | | | |
| 212131_at | DKFZP434D1335 protein | DKFZP434D1335 | Chr: 19q13.12 | 1.99 |
| 224436_s_at | DKFZp564D177 protein | DKFZp564D177 | Chr: 9q31.2 | 3.49 |
| 201681_s_at | discs, large (Drosophila) | DLG5 | Chr: 10q23 | 0.46 |
| | homolog 5 | | | |
| 209187_at | down-regulator of transcription 1, | DR1 | Chr: 1p22.1 | 1.93 |
| | TBP-binding (negative cofactor | | | |
| | 2) | | | |
| 204363_at | coagulation factor III | F3 | Chr: 1p22-p21 | 5.40 |
| | (thromboplastin, tissue factor) | | | |
| 209004_s_at | F-box and leucine-rich repeat | FBXL5 | Chr: 4p15.33 | 1.59 |
| | protein 5 | | | |
| 208933_s_at | hypothetical protein FLJ10359 | FLJ10359 | Chr: 1q42.3 | 2.98 |
| 240239_at | hypothetical protein FLJ14779 | FLJ14779 | Chr: 19q13.13 | 1.64 |
| 221518_s_at | hypothetical protein FLJ20727 | FLJ20727 | Chr: 11p15.3 | 0.59 |
| 228950_s_at | putative NFkB activating protein | FLJ23091 | Chr: 1p31.2 | 3.13 |
| | 373 | | | |
| 212558_at | ganglioside-induced | GDAP1L1 | Chr: 20q12 | 2.80 |
| | differentiation-associated protein | | | |
| | 1-like 1 | | | |
| 201864_at | GDP dissociation inhibitor 1 | GDI1 | Chr: Xq28 | 0.60 |
| 238119_at | GL004 protein | GL004 | Chr: 2q36.3 | 0.50 |
| 212294_at | guanine nucleotide binding | GNG12 | Chr: 1p31.2 | 4.08 |
| | protein (G protein), gamma 12 | | | |
| 207157_s_at | guanine nucleotide binding | GNG5 | Chr: 1p22 | 2.77 |
| | protein (G protein), gamma 5 | | | |
| 212211_at | gene trap ankyrin repeat | GTAR | Chr: 4q21.1-q21.21 | 1.43 |
| 225784_s_at | hepatocellular carcinoma- | HCA127 | Chr: Xq11.2 | 0.31 |
| | associated antigen 127 | | | |
| 223042_s_at | hepatitis C virus core-binding | HCBP6 | Chr: Xq28 | 0.66 |
| | protein 6 | | | |
| 219288_at | HT021 | HT021 | Chr: 3p21.1 | 3.24 |
| 209185_s_at | insulin receptor substrate 2 | IRS2 | Chr: 13q34 | 2.40 |
| 201464_x_at | v-jun sarcoma virus 17 oncogene | JUN | Chr: 1p32-p31 | 2.55 |
| | homolog (avian) | | | |
| 201466_s_at | v-jun sarcoma virus 17 oncogene | JUN | Chr: 1p32-p31 | 2.89 |
| | homolog (avian) | | | |
| 213340_s_at | KIAA0495 | KIAA0495 | Chr: 1p36.32 | 2.95 |
| 213271_s_at | KIAA1117 protein | KIAA1117 | Chr: 6q15 | 0.60 |
| 208935_s_at | lectin, galactoside-binding, | LGALS8 | Chr: 1q42-q43 | 2.56 |
| | soluble, 8 (galectin 8) | | | |
| 209205_s_at | LIM domain only 4 | LMO4 | Chr: 1p22.3 | 2.62 |
| 225479_at | hypothetical protein LOC116064 | LOC116064 | Chr: 3q13.33 | 0.67 |
| 227466_at | hypothetical protein LOC285550 | LOC285550 | Chr: 4p15.33 | 1.54 |
| 1558700_s_at | hypothetical protein LOC339324 | LOC339324 | Chr: 19q13.13 | 2.05 |
| 235940_at | hypothetical protein MGC10999 | MGC10999 | Chr: 9q21.33 | 5.16 |
| 228326_at | hypothetical protein MGC43690 | MGC43690 | Chr: 6q27 | 0.54 |
| 213259_s_at | similar to RIKEN cDNA | MGC9564 | Chr: 17q11.2 | 0.55 |
| | 1110002C08 gene | | | |
| 224874_at | hypothetical protein MGC9850 | MGC9850 | Chr: 13q12.2 | 3.26 |
| 212080_at | myeloid/lymphoid or mixed- | MLL | Chr: 11q23 | 0.51 |
| | lineage leukemia (trithorax | | | |
| | homolog, Drosophila) | | | |
| 208709_s_at | nardilysin (N-arginine dibasic | NRD1 | Chr: 1p32.2-p32.1 | 1.69 |
| | convertase) | | | |
| 209791_at | peptidyl arginine deiminase, type | PADI2 | Chr: 1p35.2-p35.1 | 4.02 |
| | II | | | |
| 207769_s_at | polyglutamine binding protein 1 | PQBP1 | Chr: Xp11.23 | 0.65 |
| 214527_s_at | polyglutamine binding protein 1 | PQBP1 | Chr: Xp11.23 | 0.65 |
| 224909_s_at | KIAA1415 protein | PRex1 | Chr: 20q13.13 | 2.89 |
| 208615_s_at | protein tyrosine phosphatase | PTP4A2 | Chr: 1p35 | 1.88 |
| | type IVA, member 2 | | | |
| 208616_s_at | protein tyrosine phosphatase | PTP4A2 | Chr: 1p35 | 1.87 |
| | type IVA, member 2 | | | |
| 216988_s_at | protein tyrosine phosphatase | PTP4A2 | Chr: 1p35 | 1.97 |
| | type IVA, member 2 | | | |
| 202006_at | protein tyrosine phosphatase, | PTPN12 | Chr: 7q11.23 | 1.76 |
| | non-receptor type 12 | | | |
| 201165_s_at | pumilio homolog 1 (Drosophila) | PUM1 | Chr: 1p35.2 | 1.42 |
| 225251_at | RAB24, member RAS oncogene | RAB24 | Chr: 5q35.3 | 0.53 |
| | family | | | |
| 213718_at | RNA binding motif protein 4 | RBM4 | Chr: 11q13 | 0.47 |
| 212197_x_at | Rho interacting protein 3 | RHOIP3 | Chr: 17p11.2 | 0.66 |
| 214143_x_at | ribosomal protein L24 | RPL24 | Chr: 3q12 | 0.67 |
| 211073_x_at | ribosomal protein L3 | RPL3 | Chr: 22q13 | 0.64 |
| 200717_x_at | ribosomal protein L7 | RPL7 | Chr: 8q13.3 | 0.71 |
| 200909_s_at | ribosomal protein, large P2 | RPLP2 | Chr: 11p15.5-p15.4 | 0.72 |
| 208692_at | ribosomal protein S3 | RPS3 | Chr: 11q13.3-q13.5 | 0.55 |
| 202361_at | SEC24 related gene family, | SEC24C | Chr: 10q22.3 | 0.53 |
| | member C (S. cerevisiae) | | | |
| 201696_at | splicing factor, arginine/serine- | SFRS4 | Chr: 1p35.2 | 1.48 |
| | rich 4 | | | |
| 220298_s_at | spermatogenesis associated 6 | SPATA6 | Chr: 1p33 | 5.60 |
| 238459_x_at | spermatogenesis associated 6 | SPATA6 | Chr: 1p33 | 6.03 |
| 220299_at | spermatogenesis associated 6 | SPATA6 | Chr: 1p33 | 4.09 |
| 46256_at | SPRY domain-containing SOCS | SSB3 | Chr: 16p13.3 | 0.66 |
| | box protein SSB-3 | | | |
| 209022_at | stromal antigen 2 | STAG2 | Chr: Xq25 | 0.73 |
| 201519_at | translocase of outer | TOMM70A | Chr: 3q12.3 | 0.65 |
| | mitochondrial membrane 70 | | | |
| | homolog A (yeast) | | | |
| 208661_s_at | tetratricopeptide repeat domain 3 | TTC3 | Chr: 21q22.2 | 0.46 |
| 208662_s_at | tetratricopeptide repeat domain 3 | TTC3 | Chr: 21q22.2 | 0.51 |
| 210645_s_at | tetratricopeptide repeat domain 3 | TTC3 | Chr: 21q22.2 | 0.50 |
| 219043_s_at | IAP-associated factor VIAF1 | VIAF1 | Chr: 2q12.1 | 0.78 |
| 201294_s_at | SOCS box-containing WD | WSB1 | Chr: 17q11.2 | 0.39 |
| | protein SWiP-1 | | | |
| 201296_s_at | SOCS box-containing WD | WSB1 | Chr: 17q11.2 | 0.55 |
| | protein SWiP-1 | | | |
| 207090_x_at | likely ortholog of mouse zinc | ZFP30 | Chr: 19q13.13 | 1.63 |
| | finger protein 30 | | | |
| 228157_at | zinc finger protein 207 | ZNF207 | Chr: 17q12 | 0.56 |
| 222357_at | zinc finger protein 288 | ZNF288 | Chr: 3q13.2 | 0.30 |
| 226252_at | hypothetical gene supported by | | Chr: 3q13.31 | 0.44 |
| | AK022228 | | | |
| 227388_at | hypothetical gene supported by | | Chr: 9p21.1 | 3.48 |
| | BC017510; BC036931; | | | |
| | BC028316 | | | |
| 244740_at | LOC342935 | | Chr: 19q13.43 | 1.70 |

TABLE 5
Differentially expressed probesets, which are able to discriminate on
basis of loss of heterozygosity (LOH) on the 1p locus

| | | | | ratio loss/no |
|---|---|---|---|---|
| Probe Set ID | Title | Gene Symbol | Location | loss |
| 1553954_at | hypothetical protein | MGC19780 | chr1p21.3 | 0.55 |
| | MGC19780 | | | |
| 1554433_a_at | zinc finger protein 146 | ZNF146 | chr19q13.1 | 0.57 |
| 1554479_a_at | caspase recruitment domain | CARD8 | chr19q13.32 | 0.59 |
| | family, member 8 | | | |
| 1555832_s_at | — | — | — | 0.50 |
| 1558256_at | hypothetical protein | LOC148189 | chr19q12 | 0.45 |
| | LOC148189 | | | |
| 1558604_a_at | H. sapiens mRNA; clone CD | — | — | 0.47 |
| | 43T7 | | | |
| 1558700_s_at | hypothetical protein | LOC339324 | chr19q13.12 | 0.49 |
| | LOC339324 | | | |
| 200006_at | Parkinson disease | PARK7 | chr1p36.33-p36.12 | 0.70 |
| | (autosomal recessive, early | | | |
| | onset) 7 | | | |
| 200020_at | TAR DNA binding protein | TARDBP | chr1p36.22 | 0.70 |
| 200050_at | zinc finger protein 146 | ZNF146 | chr19q13.1 | 0.52 |
| 200087_s_at | coated vesicle membrane | RNP24 | chr12q24.31 | 0.84 |
| | protein | | | |
| 200620_at | chromosome 1 open reading | C1orf8 | chr1p36-p31 | 0.58 |
| | frame 8 | | | |
| 200625_s_at | CAP, adenylate cyclase- | CAP1 | chr1p34.2 | 0.61 |
| | associated protein 1 (yeast) | | | |
| 200636_s_at | protein tyrosine phosphatase, | PTPRF | chr1p34 | 0.39 |
| | receptor type, F | | | |
| 200650_s_at | lactate dehydrogenase A | LDHA | chr11p15.4 | 0.26 |
| 200686_s_at | splicing factor, | SFRS11 | chr1p31 | 0.44 |
| | arginine/serine-rich 11 | | | |
| 200777_s_at | basic leucine zipper and W2 | BZW1 | chr2q33 | 0.75 |
| | domains 1 | | | |
| 200791_s_at | IQ motif containing GTPase | IQGAP1 | chr15q26.1 | 0.27 |
| | activating protein 1 | | | |
| 200902_at | 15 kDa selenoprotei | SEP15 | chr1p31 | 0.53 |
| 201064_s_at | poly(A) binding protein, | PABPC4 | chr1p32-p36 | 0.64 |
| | cytoplasmic 4 (inducible form) | | | |
| 201080_at | phosphatidylinositol-4- | PIP5K2B | chr17q12 | 1.63 |
| | phosphate 5-kinase, type II, | | | |
| | beta | | | |
| 201155_s_at | mitofusin 2 | MFN2 | chr1p36.22 | 0.64 |
| 201164_s_at | pumilio homolog 1 | PUM1 | chr1p35.2 | 0.52 |
| | (Drosophila) | | | |
| 201165_s_at | pumilio homolog 1 | PUM1 | chr1p35.2 | 0.74 |
| | (Drosophila) | | | |
| 201177_s_at | SUMO-1 activating enzyme | UBA2 | chr19q12 | 0.43 |
| | subunit 2 | | | |
| 201179_s_at | guanine nucleotide binding | GNAI3 | chr1p13 | 0.60 |
| | protein (G protein), alpha | | | |
| | inhibiting activity polypeptide 3 | | | |
| 201181_at | guanine nucleotide binding | GNAI3 | chr1p13 | 0.57 |
| | protein (G protein), alpha | | | |
| | inhibiting activity polypeptide 3 | | | |
| 201209_at | histone deacetylase 1 | HDAC1 | chr1p34 | 0.44 |
| 201225_s_at | serine/arginine repetitive | SRRM1 | chr1p36.11 | 0.71 |
| | matrix 1 | | | |
| 201274_at | proteasome (prosome, | PSMA5 | chr1p13 | 0.60 |
| | macropain) subunit, alpha | | | |
| | type, 5 | | | |
| 201323_at | EBNA1 binding protein 2 | EBNA1BP2 | chr1p35-p33 | 0.56 |
| 201339_s_at | sterol carrier protein 2 | SCP2 | chr1p32 | 0.56 |
| 201398_s_at | translocation associated | TRAM1 | chr8q13.3 | 0.66 |
| | membrane protein 1 | | | |
| 201426_s_at | vimentin | VIM | chr10p13 | 0.39 |
| 201445_at | calponin 3, acidic | CNN3 | chr1p22-p21 | 0.40 |
| 201519_at | translocase of outer | TOMM70A | chr3q12.2 | 1.52 |
| | mitochondrial membrane 70 | | | |
| | homolog A (yeast) | | | |
| 201667_at | gap junction protein, alpha 1, | GJA1 | chr6q21-q23.2 | 0.19 |
| | 43 kDa (connexin 43) | | | |
| 201674_s_at | A kinase (PRKA) anchor | AKAP1 | chr17q21-q23 | 1.85 |
| | protein 1 | | | |
| 201696_at | splicing factor, | SFRS4 | chr1p35.3 | 0.61 |
| | arginine/serine-rich 4 | | | |
| 201864_at | GDP dissociation inhibitor 1 | GDI1 | chrXq28 | 1.56 |
| 201948_at | guanine nucleotide binding | GNL2 | chr1p34.3 | 0.49 |
| | protein-like 2 (nucleolar) | | | |
| 202049_s_at | zinc finger protein 262 | ZNF262 | chr1p32-p34 | 0.51 |
| 202096_s_at | benzodiazapine receptor | BZRP | chr22q13.31 | 0.36 |
| | (peripheral) | | | |
| 202149_at | neural precursor cell | NEDD9 | chr6p25-p24 | 0.49 |
| | expressed, developmentally | | | |
| | down-regulated 9 | | | |
| 202250_s_at | WD repeat domain 42A | WDR42A | chr1q22-q23 | 1.78 |
| 202260_s_at | syntaxin binding protein 1 | STXBP1 | chr9q34.1 | 1.84 |
| 202299_s_at | hepatitis B virus x interacting | HBXIP | chr1p13.3 | 0.57 |
| | protein | | | |
| 202300_at | hepatitis B virus x interacting | HBXIP | chr1p13.3 | 0.59 |
| | protein | | | |
| 202361_at | SEC24 related gene family, | SEC24C | chr10q22.2 | 1.73 |
| | member C (S. cerevisiae) | | | |
| 202362_at | RAP1A, member of RAS | RAP1A | chr1p13.3 | 0.51 |
| | oncogene family | | | |
| 202412_s_at | ubiquitin specific protease 1 | USP1 | chr1p32.1-p31.3 | 0.43 |
| 202413_s_at | ubiquitin specific protease 1 | USP1 | chr1p32.1-p31.3 | 0.41 |
| 202471_s_at | isocitrate dehydrogenase 3 | IDH3G | chrXq28 | 1.54 |
| | (NAD+) gamma | | | |
| 202502_at | acyl-Coenzyme A | ACADM | chr1p31 | 0.57 |
| | dehydrogenase, C-4 to C-12 | | | |
| | straight chain | | | |
| 202625_at | v-yes-1 Yamaguchi sarcoma | LYN | chr8q13 | 0.46 |
| | viral related oncogene | | | |
| | homolog | | | |
| 202626_s_at | v-yes-1 Yamaguchi sarcoma | LYN | chr8q13 | 0.43 |
| | viral related oncogene | | | |
| | homolog | | | |
| 202668_at | ephrin-B2 | EFNB2 | chr13q33 | 0.42 |
| 202669_s_at | ephrin-B2 | EFNB2 | chr13q33 | 0.50 |
| 202868_s_at | POP4 (processing of | POP4 | chr19q12 | 0.63 |
| | precursor, S. cerevisiae) | | | |
| | homolog | | | |
| 202939_at | zinc metalloproteinase | ZMPSTE24 | chr1p34 | 0.53 |
| | (STE24 homolog, yeast) | | | |
| 202950_at | crystallin, zeta (quinone | CRYZ | chr1p31-p22 | 0.43 |
| | reductase) | | | |
| 203069_at | synaptic vesicle glycoprotein | SV2A | chr1q21.2 | 1.96 |
| | 2A | | | |
| 203221_at | transducin-like enhancer of | TLE1 | chr9q21.32 | 0.31 |
| | split 1 (E(sp1) homolog, | | | |
| | Drosophila) | | | |
| 203222_s_at | transducin-like enhancer of | TLE1 | chr9q21.32 | 0.33 |
| | split 1 (E(sp1) homolog, | | | |
| | Drosophila) | | | |
| 203283_s_at | heparan sulfate 2-O- | HS2ST1 | chr1p31.1-p22.1 | 0.29 |
| | sulfotransferase 1 | | | |
| 203284_s_at | heparan sulfate 2-O- | HS2ST1 | chr1p31.1-p22.1 | 0.51 |
| | sulfotransferase 1 | | | |
| 203288_at | KIAA0355 | KIAA0355 | chr19q13.11 | 0.60 |
| 203289_s_at | chromosome 16 open reading | C16orf35 | chr16p13.3 | 2.09 |
| | frame 35 | | | |
| 203303_at | t-complex-associated-testis- | TCTE1L | chrXp21 | 0.33 |
| | expressed 1-like | | | |
| 203310_at | syntaxin binding protein 3 | STXBP3 | chr1p13.3 | 0.48 |
| 203347_s_at | likely ortholog of mouse metal | M96 | chr1p22.1 | 0.44 |
| | response element binding | | | |
| | transcription factor 2 | | | |
| 203364_s_at | KIAA0652 gene product | KIAA0652 | chr11p11.2 | 1.59 |
| 203389_at | kinesin family member 3C | KIF3C | chr2p23 | 2.12 |
| 203401_at | phosphoribosyl | PRPS2 | chrXp22.3-p22.2 | 0.35 |
| | pyrophosphate synthetase 2 | | | |
| 203511_s_at | trafficking protein particle | TRAPPC3 | chr1p34.3 | 0.55 |
| | complex 3 | | | |
| 203560_at | gamma-glutamyl hydrolase | GGH | chr8q12.3 | 0.37 |
| | (conjugase, | | | |
| | folylpolygammaglutamyl | | | |
| | hydrolase) | | | |
| 203611_at | telomeric repeat binding | TERF2 | chr16q22.1 | 1.59 |
| | factor 2 | | | |
| 203765_at | grancalcin, EF-hand calcium | GCA | chr2q24.2 | 0.32 |
| | binding protein | | | |
| 203787_at | single-stranded DNA binding | SSBP2 | chr5q14.1 | 0.38 |
| | protein 2 | | | |
| 203819_s_at | IGF-II mRNA-binding protein 3 | IMP-3 | chr7p11 | 0.05 |
| 203928_x_at | microtubule-associated | MAPT | chr17q21.1 | 2.57 |
| | protein tau | | | |
| 203930_s_at | microtubule-associated | MAPT | chr17q21.1 | 2.44 |
| | protein tau | | | |
| 204011_at | sprouty homolog 2 | SPRY2 | chr13q31.1 | 0.33 |
| | (Drosophila) | | | |
| 204022_at | Nedd-4-like ubiquitin-protein | WWP2 | chr16q22.1 | 1.93 |
| | ligase | | | |
| 204036_at | endothelial differentiation, | EDG2 | chr9q31.3 | 0.15 |
| | lysophosphatidic acid G- | | | |
| | protein-coupled receptor, 2 | | | |
| 204228_at | peptidyl prolyl isomerase H | PPIH | chr1p34.1 | 0.49 |
| | (cyclophilin H) | | | |
| 204299_at | FUS interacting protein | FUSIP1 | chr1p36.11 | 0.52 |
| | (serine-arginine rich) 1 | | | |
| 204363_at | coagulation factor III | F3 | chr1p22-p21 | 0.16 |
| | (thromboplastin, tissue factor) | | | |
| 204379_s_at | fibroblast growth factor | FGFR3 | chr4p16.3 | 0.20 |
| | receptor 3 (achondroplasia, | | | |
| | thanatophoric dwarfism) | | | |
| 204400_at | embryonal Fyn-associated | EFS | chr14q11.2-q12 | 2.55 |
| | substrate | | | |
| 204451_at | frizzled homolog 1 | FZD1 | chr7q21 | 0.38 |
| | (Drosophila) | | | |
| 204722_at | sodium channel, voltage- | SCN3B | chr11q24.1 | 4.57 |
| | gated, type II, beta | | | |
| 204984_at | glypican 4 | GPC4 | chrXq26.1 | 0.40 |
| 205095_s_at | ATPase, H+ transporting, | ATP6V0A1 | chr17q21 | 1.80 |
| | lysosomal V0 subunit a | | | |
| | isoform 1 | | | |
| 205130_at | renal tumor antigen | RAGE | chr14q32 | 0.52 |
| 205140_at | fucose-1-phosphate | FPGT | chr1p31.1 | 0.36 |
| | guanylytransferase | | | |
| 205173_x_at | CD58 antigen, (lymphocyte | CD58 | chr1p13 | 0.22 |
| | function-associated antigen | | | |
| | 3) | | | |
| 205176_s_at | integrin beta 3 binding protein | ITGB3BP | chr1p31.3 | 0.48 |
| | (beta3-endonexin) | | | |
| 205260_s_at | acylphosphatase 1, | ACYP1 | chr14q24.3 | 0.44 |
| | erythrocyte (common) type | | | |
| 205263_at | B-cell CLL/lymphoma 10 | BCL10 | chr1p22 | 0.54 |
| 205292_s_at | heterogeneous nuclear | HNRPA2B1 | chr7p15 | 0.72 |
| | ribonucleoprotein A2/B1 | | | |
| 205497_at | zinc finger protein 175 | ZNF175 | chr19q13.4 | 0.63 |
| 205852_at | cyclin-dependent kinase 5, | CDK5R2 | chr2q35 | 2.45 |
| | regulatory subunit 2 (p39) | | | |
| 205996_s_at | adenylate kinase 2 | AK2 | chr1p34 | 0.59 |
| 206095_s_at | FUS interacting protein | FUSIP1 | chr1p36.11 | 0.43 |
| | (serine-arginine rich) 1 | | | |
| 206401_s_at | microtubule-associated | MAPT | chr17q21.1 | 2.72 |
| | protein tau | | | |
| 206993_at | ATP synthase, H+ | ATP5S | chr14q21.3 | 1.42 |
| | transporting, mitochondrial F0 | | | |
| | complex, subunit s (factor B) | | | |
| 207090_x_at | zinc finger protein KIAA0961 | KIAA0961 | chr19q13.13 | 0.61 |
| 207236_at | zinc finger protein 345 | ZNF345 | chr19q13.12 | 0.45 |
| 207358_x_at | microtubule-actin crosslinking | MACF1 | chr1p32-p31 | 0.54 |
| | factor 1 | | | |
| 208095_s_at | signal recognition particle | SRP72 | chr4q11 | 0.66 |
| | 72 kDa | | | |
| 208374_s_at | capping protein (actin | CAPZA1 | chr1p13.2 | 0.55 |
| | filament) muscle Z-line, alpha 1 | | | |
| 208615_s_at | protein tyrosine phosphatase | PTP4A2 | chr1p35 | 0.51 |
| | type IVA, member 2 | | | |
| 208680_at | peroxiredoxin 1 | PRDX1 | chr1p34.1 | 0.33 |
| 208709_s_at | nardilysin (N-arginine dibasic | NRD1 | chr1p32.2-p32.1 | 0.60 |
| | convertase) | | | |
| 208723_at | ubiquitin specific protease 11 | USP11 | chrXp11.23 | 1.92 |
| 208728_s_at | cell division cycle 42 (GTP | CDC42 | chr1p36.1 | 0.55 |
| | binding protein, 25 kDa) | | | |
| 208766_s_at | heterogeneous nuclear | HNRPR | chr1p36.12 | 0.67 |
| | ribonucleoprotein R | | | |
| 208924_at | ring finger protein 11 | RNF11 | chr1pter-p22.1 | 0.65 |
| 208971_at | uroporphyrinogen | UROD | chr1p34 | 0.63 |
| | decarboxylase | | | |
| 209001_s_at | anaphase promoting complex | ANAPC13 | chr3q22.1 | 1.32 |
| | subunit 13 | | | |
| 209045_at | X-prolyl aminopeptidase | XPNPEP1 | chr10q25.3 | 1.44 |
| | (aminopeptidase P) 1, soluble | | | |
| 209099_x_at | jagged 1 (Alagille syndrome) | JAG1 | chr20p12.1-p11.23 | 0.38 |
| 209117_at | WW domain binding protein 2 | WBP2 | chr17q25 | 2.05 |
| 209120_at | nuclear receptor subfamily 2, | NR2F2 | chr15q26 | 0.33 |
| | group F, member 2 | | | |
| 209187_at | down-regulator of | DR1 | chr1p22.1 | 0.46 |
| | transcription 1, TBP-binding | | | |
| | (negative cofactor 2) | | | |
| 209355_s_at | phosphatidic acid | PPAP2B | chr1pter-p22.1 | 0.25 |
| | phosphatase type 2B | | | |
| 209537_at | exostoses (multiple)-like 2 | EXTL2 | chr1p21 | 0.61 |
| 209669_s_at | PAI-1 mRNA-binding protein | PAI-RBP1 | chr1p31-p22 | 0.48 |
| 209707_at | phosphatidylinositol glycan, | PIGK | chr1p31.1 | 0.62 |
| | class K | | | |
| 209711_at | solute carrier family 35 (UDP- | SLC35D1 | chr1p32-p31 | 0.49 |
| | glucuronic acid/UDP-N- | | | |
| | acetylgalactosamine dual | | | |
| | transporter), member D1 | | | |
| 209875_s_at | secreted phosphoprotein 1 | SPP1 | chr4q21-q25 | 0.22 |
| | (osteopontin, bone | | | |
| | sialoprotein I, early T- | | | |
| | lymphocyte activation 1) | | | |
| 210092_at | mago-nashi homolog, | MAGOH | chr1p34-p33 | 0.40 |
| | proliferation-associated | | | |
| | (Drosophila) | | | |
| 210093_s_at | mago-nashi homolog, | MAGOH | chr1p34-p33 | 0.51 |
| | proliferation-associated | | | |
| | (Drosophila) | | | |
| 210137_s_at | dCMP deaminase | DCTD | chr4q35.1 | 0.17 |
| 210178_x_at | FUS interacting protein | FUSIP1 | chr1p36.11 | 0.54 |
| | (serine-arginine rich) 1 | | | |
| 210191_s_at | putative homeodomain | PHTF1 | chr1p13 | 0.65 |
| | transcription factor 1 | | | |
| 210371_s_at | retinoblastoma binding | RBBP4 | chr1p35.1 | 0.48 |
| | protein 4 | | | |
| 210502_s_at | peptidylprolyl isomerase E | PPIE | chr1p32 | 0.54 |
| | (cyclophilin E) | | | |
| 210517_s_at | A kinase (PRKA) anchor | AKAP12 | chr6q24-q25 | 0.36 |
| | protein (gravin) 12 | | | |
| 210645_s_at | tetratricopeptide repeat | TTC3 | chr21q22.2 | 1.94 |
| | domain 3 | | | |
| 210754_s_at | v-yes-1 Yamaguchi sarcoma | LYN | chr8q13 | 0.57 |
| | viral related oncogene | | | |
| | homolog | | | |
| 210770_s_at | calcium channel, voltage- | CACNA1A | chr19p13.2-p13.1 | 3.12 |
| | dependent, P/Q type, alpha | | | |
| | 1A subunit | | | |
| 210829_s_at | single-stranded DNA binding | SSBP2 | chr5q14.1 | 0.33 |
| | protein 2 | | | |
| 210840_s_at | IQ motif containing GTPase | IQGAP1 | chr15q26.1 | 0.32 |
| | activating protein 1 | | | |
| 211383_s_at | WD repeat domain 37 | WDR37 | chr10p15.3 | 1.34 |
| 211474_s_at | serine (or cysteine) | SERPINB6 | chr6p25 | 0.54 |
| | proteinase inhibitor, clade B | | | |
| | (ovalbumin), member 6 | | | |
| 211488_s_at | integrin, beta 8 | ITGB8 | chr7p21.1 | 0.55 |
| 211662_s_at | voltage-dependent anion | VDAC2 | chr10q22 | 1.62 |
| | channel 2 | | | |
| 211703_s_at | beta-amyloid binding protein | BBP | chr1p31.3 | 0.44 |
| | precursor | | | |
| 211733_x_at | sterol carrier protein 2 | SCP2 | chr1p32 | 0.64 |
| 211755_s_at | ATP synthase, H+ | ATP5F1 | chr1p13.2 | 0.67 |
| | transporting, mitochondrial F0 | | | |
| | complex, subunit b, isoform 1 | | | |
| 212131_at | family with sequence | FAM61A | chr19q13.11 | 0.48 |
| | similarity 61, member A | | | |
| 212132_at | family with sequence | FAM61A | chr19q13.11 | 0.35 |
| | similarity 61, member A | | | |
| 212192_at | potassium channel | KCTD12 | chr13q22.3 | 0.36 |
| | tetramerisation domain | | | |
| | containing 12 | | | |
| 212226_s_at | phosphatidic acid | PPAP2B | chr1pter-p22.1 | 0.33 |
| | phosphatase type 2B | | | |
| 212230_at | phosphatidic acid | PPAP2B | chr1pter-p22.1 | 0.32 |
| | phosphatase type 2B | | | |
| 212245_at | multiple coagulation factor | MCFD2 | chr2p21 | 0.66 |
| | deficiency 2 | | | |
| 212294_at | guanine nucleotide binding | GNG12 | chr1p31.2 | 0.24 |
| | protein (G protein), gamma | | | |
| | 12 | | | |
| 212355_at | KIAA0323 protein | KIAA0323 | chr14q11.2 | 0.49 |
| 212370_x_at | family with sequence | FAM21B | chr10q11.22 /// | 1.61 |
| | similarity 21, member B | | chr10q11.23 | |
| 212383_at | ATPase, H+ transporting, | ATP6V0A1 | chr17q21 | 1.74 |
| | lysosomal V0 subunit a | | | |
| | isoform 1 | | | |
| 212393_at | SET binding factor 1 | SBF1 | chr22q13.33 | 1.77 |
| 212491_s_at | DnaJ (Hsp40) homolog, | DNAJC8 | chr1p35.3 | 0.56 |
| | subfamily C, member 8 | | | |
| 212503_s_at | KIAA0934 protein | KIAA0934 | chr10p15.3 | 1.29 |
| 212513_s_at | ubiquitin specific protease 33 | USP33 | chr1p31.1 | 0.53 |
| 212515_s_at | DEAD (Asp-Glu-Ala-Asp) box | DDX3X | chrXp11.3-p11.23 | 0.74 |
| | polypeptide 3, X-linked | | | |
| 212628_at | Protein kinase N2 | PKN2 | chr1p22.2 | 0.47 |
| 212698_s_at | septin 10 | SEPT10 | chr2q13 | 0.43 |
| 212699_at | secretory carrier membrane | SCAMP5 | chr15q23 | 2.34 |
| | protein 5 | | | |
| 212893_at | zinc finger, ZZ domain | ZZZ3 | chr1p31.1 | 0.49 |
| | containing 3 | | | |
| 212920_at | Homo sapiens transcribed | — | — | 0.40 |
| | sequence with weak | | | |
| | similarity to protein | | | |
| | ref: NP_060312.1 (H. sapiens) | | | |
| | hypothetical protein | | | |
| | FLJ20489 [Homo sapiens] | | | |
| 212928_at | TSPY-like 4 | TSPYL4 | chr6q22.1 | 1.41 |
| 213001_at | angiopoietin-like 2 | ANGPTL2 | chr9q34 | 3.95 |
| 213004_at | angiopoietin-like 2 | ANGPTL2 | chr9q34 | 4.16 |
| 213156_at | Homo sapiens mRNA; cDNA | — | — | 1.89 |
| | DKFZp586B211 (from clone | | | |
| | DKFZp586B211) | | | |
| 213158_at | Homo sapiens mRNA; cDNA | — | — | 1.66 |
| | DKFZp586B211 (from clone | | | |
| | DKFZp586B211) | | | |
| 213170_at | glutathione peroxidase 7 | GPX7 | chr1p32 | 0.52 |
| 213186_at | zinc finger DAZ interacting | DZIP3 | chr3q13.13 | 1.48 |
| | protein 3 | | | |
| 213259_s_at | sterile alpha and TIR motif | SARM1 | chr17q11 | 1.87 |
| | containing 1 | | | |
| 213340_s_at | KIAA0495 | KIAA0495 | chr1p36.32 | 0.35 |
| 213351_s_at | transmembrane and coiled- | TMCC1 | chr3q21.3 | 1.62 |
| | coil domains 1 | | | |
| 213424_at | KIAA0895 protein | KIAA0895 | chr7p14.1 | 0.66 |
| 213436_at | cannabinoid receptor 1 | CNR1 | chr6q14-q15 | 0.32 |
| | (brain) | | | |
| 213439_x_at | RaP2 interacting protein 8 | RPIP8 | chr17q21.31 | 2.77 |
| 213464_at | SHC (Src homology 2 domain | SHC2 | chr19p13.3 | 2.03 |
| | containing) transforming | | | |
| | protein 2 | | | |
| 213467_at | FALSE | — | — | 2.59 |
| 213557_at | CDC2-related protein kinase 7 | CRK7 | chr17q12 | 1.67 |
| 213798_s_at | CAP, adenylate cyclase- | CAP1 | chr1p34.2 | 0.57 |
| | associated protein 1 (yeast) | | | |
| 213883_s_at | beta-amyloid binding protein | BBP | chr1p31.3 | 0.52 |
| | precursor | | | |
| 214241_at | NADH dehydrogenase | NDUFB8 | chr10q23.2-q23.33 | 1.66 |
| | (ubiquinone) 1 beta | | | |
| | subcomplex, 8, 19 kDa | | | |
| 214383_x_at | kelch domain containing 3 | KLHDC3 | chr6p21.1 | 1.44 |
| 214894_x_at | microtubule-actin crosslinking | MACF1 | chr1p32-p31 | 0.58 |
| | factor 1 | | | |
| 214933_at | calcium channel, voltage- | CACNA1A | chr19p13.2-p13.1 | 2.72 |
| | dependent, P/Q type, alpha | | | |
| | 1A subunit | | | |
| 215017_s_at | formin binding protein 1-like | FNBP1L | chr1p22.1 | 0.20 |
| 215222_x_at | microtubule-actin crosslinking | MACF1 | chr1p32-p31 | 0.50 |
| | factor 1 | | | |
| 215691_x_at | chromosome 1 open reading | C1orf41 | chr1p32.1-p33 | 0.44 |
| | frame 41 | | | |
| 216268_s_at | jagged 1 (Alagille syndrome) | JAG1 | chr20p12.1-p11.23 | 0.39 |
| 216903_s_at | calcium binding atopy-related | CBARA1 | chr10q22.1 | 1.67 |
| | autoantigen 1 | | | |
| 217724_at | PAI-1 mRNA-binding protein | PAI-RBP1 | chr1p31-p22 | 0.67 |
| 217877_s_at | hypothetical protein SP192 | SP192 | chr1p34.1 | 0.44 |
| 217893_s_at | hypothetical protein | FLJ12666 | chr1p34.3 | 0.50 |
| | FLJ12666 | | | |
| 217921_at | | — | — | 0.56 |
| 217968_at | tumor suppressing | TSSC1 | chr2p25.2 | 1.52 |
| | subtransferable candidate 1 | | | |
| 218011_at | ubiquitin-like 5 | UBL5 | chr19p13.3 | 0.57 |
| 218097_s_at | CUE domain containing 2 | CUEDC2 | chr10q24.32 | 1.46 |
| 218302_at | presenilin enhancer 2 | PSENEN | chr19q13.12 | 0.50 |
| | homolog (C. elegans) | | | |
| 218370_s_at | hypothetical protein | FLJ12903 | chr1p35.1 | 0.61 |
| | FLJ12903 | | | |
| 218462_at | RNA processing factor 1 | RPF1 | chr1p22.3 | 0.44 |
| 218490_s_at | zinc finger protein 302 | ZNF302 | chr19q13.11 | 0.49 |
| 218577_at | hypothetical protein | FLJ20331 | chr1p31.1 | 0.62 |
| | FLJ20331 | | | |
| 218640_s_at | pleckstrin homology domain | PLEKHF2 | chr8q22.1 | 0.32 |
| | containing, family F (with | | | |
| | FYVE domain) member 2 | | | |
| 218712_at | hypothetical protein | FLJ20508 | chr1p34.3 | 0.48 |
| | FLJ20508 | | | |
| 218924_s_at | chitobiase, di-N-acetyl- | CTBS | chr1p22 | 0.37 |
| 218938_at | F-box and leucine-rich repeat | FBXL15 | chr10q24.32 | 2.59 |
| | protein 15 | | | |
| 219094_at | armadillo repeat containing 8 | ARMC8 | chr3q22.3 | 1.53 |
| 219314_s_at | zinc finger protein 219 | ZNF219 | chr14q11 | 1.88 |
| 219372_at | carnitine deficiency- | CDV1 | chr12q24.13 | 0.60 |
| | associated, expressed in | | | |
| | ventricle 1 | | | |
| 219375_at | choline/ethanolaminephospho | CEPT1 | chr1p13.3 | 0.58 |
| | transferase | | | |
| 219494_at | RAD54B homolog | RAD54B | chr8q21.3-q22 | 0.34 |
| 219818_s_at | G patch domain containing 1 | GPATC1 | chr19q13.11 | 0.52 |
| 219848_s_at | zinc finger protein 432 | ZNF432 | chr19q13.41 | 0.53 |
| 219939_s_at | upstream of NRAS | UNR | chr1p22 | 0.65 |
| 220358_at | Jun dimerization protein | SNFT | chr1q32.3 | 0.47 |
| | p21SNFT | | | |
| 220443_s_at | ventral anterior homeobox 2 | VAX2 | chr2p13 | 2.58 |
| 221024_s_at | solute carrier family 2 | SLC2A10 | chr20q13.1 | 0.09 |
| | (facilitated glucose | | | |
| | transporter), member 10 | | | |
| 221432_s_at | solute carrier family 25, | SLC25A28 | chr10q23-q24 | 1.72 |
| | member 28 | | | |
| 221486_at | endosulfine alpha | ENSA | chr1q21.2 | 1.66 |
| 221522_at | ankyrin repeat domain 27 | ANKRD27 | chr19q13.11 | 0.62 |
| | (VPS9 domain) | | | |
| 221679_s_at | abhydrolase domain | ABHD6 | chr3p14.3 | 1.90 |
| | containing 6 | | | |
| 221958_s_at | putative NFkB activating | FLJ23091 | chr1p31.2 | 0.35 |
| | protein 373 | | | |
| 222409_at | coronin, actin binding protein, | CORO1C | chr12q24.1 | 1.60 |
| | 1C | | | |
| 222452_s_at | hypothetical protein SP192 | SP192 | chr1p34.1 | 0.50 |
| 222459_at | hypothetical protein | FLJ12666 | chr1p34.3 | 0.59 |
| | FLJ12666 | | | |
| 222495_at | protein x 013 | AD-020 | chr1p13.3 | 0.54 |
| 222580_at | zinc finger protein 644 | ZNF644 | chr1p22.2 | 0.57 |
| 222654_at | myo-inositol | IMPA3 | chr8q12.1 | 0.60 |
| | monophosphatase A3 | | | |
| 222699_s_at | pleckstrin homology domain | PLEKHF2 | chr8q22.1 | 0.34 |
| | containing, family F (with | | | |
| | FYVE domain) member 2 | | | |
| 222833_at | hypothetical protein | FLJ20481 | chr16q12.2 | 0.25 |
| | FLJ20481 | | | |
| 222834_s_at | guanine nucleotide binding | GNG12 | chr1p31.2 | 0.40 |
| | protein (G protein), gamma | | | |
| | 12 | | | |
| 222893_s_at | hypothetical protein | FLJ13150 | chr1p22.1 | 0.55 |
| | FLJ13150 | | | |
| 222975_s_at | upstream of NRAS | UNR | chr1p22 | 0.62 |
| 223017_at | endoplasmic reticulum | TLP19 | chr1p32.3 | 0.48 |
| | thioredoxin superfamily | | | |
| | member, 18 kDa | | | |
| 223042_s_at | FUN14 domain containing 2 | FUNDC2 | chrXq28 | 1.47 |
| 223066_at | SNARE associated protein | SNAPAP | chr1q21.3 | 0.65 |
| | snapin | | | |
| 223103_at | START domain containing 10 | STARD10 | chr11q13 | 2.40 |
| 223120_at | fucosidase, alpha-L-2, | FUCA2 | chr6q24 | 0.34 |
| | plasma | | | |
| 223125_s_at | chromosome 1 open reading | C1orf21 | chr1q25 | 0.51 |
| | frame 21 | | | |
| 223132_s_at | tripartite motif-containing 8 | TRIM8 | chr10q24.3 | 1.84 |
| 223159_s_at | NIMA (never in mitosis gene | NEK6 | chr9q33.3-q34.11 | 0.36 |
| | a)-related kinase 6 | | | |
| 223230_at | hypothetical protein | FLJ14936 | chr1p33-p32.1 | 0.58 |
| | FLJ14936 | | | |
| 223296_at | mitochondrial carrier protein | MGC4399 | chr1p36.22 | 0.65 |
| 223331_s_at | DEAD (Asp-Glu-Ala-Asp) box | DDX20 | chr1p21.1-p13.2 | 0.53 |
| | polypeptide 20 | | | |
| 223398_at | chromosome 9 open reading | C9orf89 | chr9q22.31 | 0.22 |
| | frame 89 | | | |
| 223418_x_at | hypothetical protein | DKFZP566D1346 | chr1p32.3-p31.3 | 0.58 |
| | DKFZp566D1346 | | | |
| 223435_s_at | protocadherin alpha | PCDHA9 /// | chr5q31 | 2.25 |
| | 9///protocadherin alpha | PCDHAC2 /// | | |
| | subfamily C, 2///protocadherin | PCDHAC1 /// | | |
| | alpha subfamily C, | PCDHA13 /// | | |
| | 1///protocadherin alpha | PCDHA12 /// | | |
| | 13///protocadherin alpha | PCDHA11 /// | | |
| | 12///protocadherin alpha | PCDHA10 /// | | |
| | 11///protocadherin alpha | PCDHA8 /// | | |
| | 10///protocadherin alpha | PCDHA7 /// | | |
| | 8///protocadherin alpha | PCDHA6 /// | | |
| | 7///protocadherin alpha | PCDHA5 /// | | |
| | 6///protocadherin alpha | PCDHA4 /// | | |
| | 5///protocadherin alpha | PCDHA3 /// | | |
| | 4///protocadherin alpha | PCDHA2 /// | | |
| | 3///protocadherin alpha | PCDHA1 | | |
| | 2///protocadherin alpha 1 | | | |
| 223500_at | complexin 1 | CPLX1 | chr4p16.3 | 3.79 |
| 223603_at | zinc finger protein 179 | ZNF179 | chr17p11.2 | 2.71 |
| 223824_at | chromosome 10 open reading | C10orf59 | chr10q23.31 | 0.60 |
| | frame 59 | | | |
| 224212_s_at | protocadherin alpha | PCDHA9 /// | chr5q31 | 2.12 |
| | 9///protocadherin alpha | PCDHAC2 /// | | |
| | subfamily C, 2///protocadherin | PCDHAC1 /// | | |
| | alpha subfamily C, | PCDHA13 /// | | |
| | 1///protocadherin alpha | PCDHA12 /// | | |
| | 13///protocadherin alpha | PCDHA11 /// | | |
| | 12///protocadherin alpha | PCDHA10 /// | | |
| | 11///protocadherin alpha | PCDHA8 /// | | |
| | 10///protocadherin alpha | PCDHA7 /// | | |
| | 8///protocadherin alpha | PCDHA6 /// | | |
| | 7///protocadherin alpha | PCDHA5 /// | | |
| | 6///protocadherin alpha | PCDHA4 /// | | |
| | 5///protocadherin alpha | PCDHA3 /// | | |
| | 4///protocadherin alpha | PCDHA2 /// | | |
| | 3///protocadherin alpha | PCDHA1 | | |
| | 2///protocadherin alpha 1 | | | |
| 224280_s_at | hypothetical protein RP1- | LOC56181 | chr1p36.11 | 0.49 |
| | 317E23 | | | |
| 224315_at | DEAD (Asp-Glu-Ala-Asp) box | DDX20 | chr1p21.1-p13.2 | 0.58 |
| | polypeptide 20 | | | |
| 224565_at | trophoblast-derived | TncRNA | chr11q13.1 | 0.32 |
| | noncoding RNA | | | |
| 224591_at | HP1-BP74 | HP1-BP74 | chr1p36.12 | 0.60 |
| 224686_x_at | Homo sapiens transcribed | — | chr17q21.32 | 1.47 |
| | sequence with strong | | | |
| | similarity to protein | | | |
| | ref: NP_060471.1 (H. sapiens) | | | |
| | hypothetical protein | | | |
| | FLJ10120 [Homo sapiens] | | | |
| 224867_at | similar to protein of fungal | LOC440574 | chr1p36.13 | 0.51 |
| | metazoan origin like (11.1 kD) | | | |
| | (2C514) | | | |
| 224909_s_at | KIAA1415 protein | PREX1 | chr20q13.13 | 0.37 |
| 224925_at | KIAA1415 protein | PREX1 | chr20q13.13 | 0.34 |
| 224937_at | prostaglandin F2 receptor | PTGFRN | chr1p13.1 | 0.44 |
| | negative regulator | | | |
| 224985_at | neuroblastoma RAS viral (v- | NRAS | chr1p13.2 | 0.63 |
| | ras) oncogene homolog | | | |
| 225222_at | hippocampus abundant gene | HIAT1 | chr1p21.3 | 0.58 |
| | transcript 1 | | | |
| 225327_at | hypothetical protein | FLJ10980 | chr15q21.2-q21.3 | 1.81 |
| | FLJ10980 | | | |
| 225379_at | microtubule-associated | MAPT | chr17q21.1 | 1.89 |
| | protein tau | | | |
| 225382_at | zinc finger protein 275 | ZNF275 | chrXq28 | 2.37 |
| 225384_at | dedicator of cytokinesis 7 | DOCK7 | chr1p31.3 | 0.40 |
| 225475_at | mesoderm induction early | MI-ER1 | chr1p31.2 | 0.51 |
| | response 1 | | | |
| 225479_at | CDNA FLJ32247 fis, clone | — | — | 1.46 |
| | PROST1000120 | | | |
| 225612_s_at | UDP-GlcNAc:betaGal beta- | B3GNT5 | chr3q28 | 0.30 |
| | 1,3-N- | | | |
| | acetylglucosaminyltransferase 5 | | | |
| 225633_at | hypothetical protein | LOC147991 | chr19q13.11 | 0.64 |
| | LOC147991 | | | |
| 225878_at | kinesin family member 1B | KIF1B | chr1p36.2 | 0.59 |
| 225925_s_at | ubiquitin specific protease 48 | USP48 | chr1p36.12 | 0.58 |
| 226000_at | hypothetical protein | DKFZp547A023 | chr1p13.2 | 0.43 |
| | DKFZp547A023 | | | |
| 226116_at | Homo sapiens cDNA | — | — | 0.72 |
| | FLJ12540 fis, clone | | | |
| | NT2RM4000425. | | | |
| 226189_at | Homo sapiens, clone | — | — | 0.46 |
| | IMAGE: 4794726, mRNA | | | |
| 226294_x_at | hypothetical protein | FLJ23790 | chr8q24.13 | 0.70 |
| | FLJ23790 | | | |
| 226411_at | ecotropic viral integration site | EVI5L | chr19p13.2 | 2.15 |
| | 5-like | | | |
| 226458_at | Homo sapiens, clone | — | — | 0.53 |
| | IMAGE: 4449283, mRNA | | | |
| 226487_at | hypothetical protein | FLJ14721 | chr12q24.11 | 3.21 |
| | FLJ14721 | | | |
| 226517_at | branched chain | BCAT1 | chr12pter-q12 | 0.17 |
| | aminotransferase 1, cytosolic | | | |
| 226532_at | Homo sapiens transcribed | — | — | 0.49 |
| | sequence with weak | | | |
| | similarity to protein | | | |
| | ref: NP_055301.1 (H. sapiens) | | | |
| | neuronal thread protein | | | |
| | [Homo sapiens] | | | |
| 226601_at | solute carrier family 30 (zinc | SLC30A7 | chr1p21.2 | 0.65 |
| | transporter), member 7 | | | |
| 226630_at | chromosome 14 open reading | C14orf106 | chr14q21.3 | 0.49 |
| | frame 106 | | | |
| 226760_at | hypothetical protein | LOC203411 | chrXp22.13 | 1.38 |
| | LOC203411 | | | |
| 226909_at | KIAA1729 protein | KIAA1729 | chr4p16.1 | 0.20 |
| 226976_at | Karyopherin alpha 6 (importin | KPNA6 | chr1p35.1-p34.3 | 0.55 |
| | alpha 7) | | | |
| 227081_at | dynein, axonemal, light | DNALI1 | chr1p35.1 | 0.34 |
| | intermediate polypeptide 1 | | | |
| 227091_at | KIAA1505 protein | KIAA1505 | chr7p12.3 | 0.59 |
| 227112_at | — | — | — | 1.96 |
| 227154_at | hypothetical protein | MGC15730 | chr1p36.13 | 2.74 |
| | MGC15730 | | | |
| 227199_at | Chromosome 21 open | C21orf106 | chr21q22.3 | 1.53 |
| | reading frame 106 | | | |
| 227222_at | F-box only protein 10 | FBXO10 | chr9p13.2 | 1.73 |
| 227270_at | hypothetical protein | LOC285550 | chr4p15.33 | 0.47 |
| | LOC285550 | | | |
| 227278_at | Homo sapiens transcribed | — | — | 0.48 |
| | sequence with weak | | | |
| | similarity to protein | | | |
| | ref: NP_055301.1 (H. sapiens) | | | |
| | neuronal thread protein | | | |
| | [Homo sapiens] | | | |
| 227334_at | ubiquitin specific protease 54 | USP54 | chr10q22.2 | 2.18 |
| 227361_at | heparan sulfate | HS3ST3B1 | chr17p12-p11.2 | 0.08 |
| | (glucosamine) 3-O- | | | |
| | sulfotransferase 3B1 | | | |
| 227388_at | tumor suppressor candidate 1 | TUSC1 | chr9p21.1 | 0.39 |
| 227449_at | EPH receptor A4 | EPHA4 | chr2q36.1 | 0.32 |
| 227456_s_at | chromosome 6 open reading | C6orf136 | chr6p21.33 | 1.59 |
| | frame 136 | | | |
| 227573_s_at | KIAA0657 protein | KIAA0657 | chr2q35 | 1.71 |
| 227639_at | phosphatidylinositol glycan, | PIGK | chr1p31.1 | 0.51 |
| | class K | | | |
| 227674_at | zinc finger protein 585A | ZNF585A | chr19q13.12 | 0.32 |
| 227680_at | zinc finger protein 326 | ZNF326 | chr1p22.2 | 0.56 |
| 227812_at | tumor necrosis factor receptor | TNFRSF19 | chr13q12.11-q12.3 | 0.25 |
| | superfamily, member 19 | | | |
| 227845_s_at | src homology 2 domain- | SHD | chr19p13.3 | 5.98 |
| | containing transforming | | | |
| | protein D | | | |
| 227889_at | hypothetical protein | FLJ20481 | chr16q12.2 | 0.40 |
| | FLJ20481 | | | |
| 227898_s_at | hypothetical protein | FLJ38705 | chr8q24.3 | 1.73 |
| | FLJ38705 | | | |
| 228020_at | hypothetical protein | FLJ20758 | chr2p11.2 | 1.64 |
| | FLJ20758 | | | |
| 228135_at | chromosome 1 open reading | C1orf52 | chr1p22.3 | 0.52 |
| | frame 52 | | | |
| 228165_at | hypothetical protein | DKFZp547D2210 | chr12p13.31 | 2.36 |
| | DKFZp547D2210 | | | |
| 228190_at | — | — | — | 0.43 |
| 228284_at | transducin-like enhancer of | TLE1 | chr9q21.32 | 0.45 |
| | split 1 (E(sp1) homolog, | | | |
| | Drosophila) | | | |
| 228415_at | adaptor-related protein | AP1S2 | chrXp22.2 | 0.35 |
| | complex 1, sigma 2 subunit | | | |
| 228422_at | Homo sapiens, clone | — | — | 2.08 |
| | IMAGE: 5300488, mRNA | | | |
| 228538_at | zinc finger protein 662 | ZNF662 | chr3p22.1 | 0.33 |
| 228600_x_at | hypothetical protein | MGC72075 | chr7p15.3 | 0.12 |
| | MGC72075 | | | |
| 228652_at | hypothetical protein | FLJ38288 | chr19q13.43 | 0.55 |
| | FLJ38288 | | | |
| 228730_s_at | secernin 2 | SCRN2 | chr17q21.32 | 1.63 |
| 228805_at | FLJ44216 protein | FLJ44216 | chr5q35.2 | 0.41 |
| 228813_at | histone deacetylase 4 | HDAC4 | chr2q37.2 | 2.68 |
| 228949_at | putative NFkB activating | FLJ23091 | chr1p31.2 | 0.30 |
| | protein 373 | | | |
| 228950_s_at | putative NFkB activating | FLJ23091 | chr1p31.2 | 0.40 |
| | protein 373 | | | |
| 228970_at | archease | ARCH | chr1p35.1 | 0.54 |
| 229228_at | cAMP responsive element | CREB5 | chr7p15.1 | 0.34 |
| | binding protein 5 | | | |
| 229299_at | hypothetical protein | FLJ30596 | chr5p13.2 | 0.35 |
| | FLJ30596 | | | |
| 229318_at | Homo sapiens transcribed | — | — | 1.71 |
| | sequences | | | |
| 229435_at | GLIS family zinc finger | GLIS3 | chr9p24.2 | 0.20 |
| 229498_at | Homo sapiens transcribed | — | — | 0.29 |
| | sequences | | | |
| 230258_at | GLIS family zinc finger | GLIS3 | chr9p24.2 | 0.34 |
| 230350_at | Homo sapiens transcribed | — | — | 1.87 |
| | sequence with moderate | | | |
| | similarity to protein | | | |
| | ref: NP_060312.1 (H. sapiens) | | | |
| | hypothetical protein | | | |
| | FLJ20489 [Homo sapiens] | | | |
| 230352_at | Phosphoribosyl | PRPS2 | chrXp22.3-p22.2 | 0.25 |
| | pyrophosphate synthetase 2 | | | |
| 230637_at | sideroflexin 4 | SFXN4 | chr10q26.11 | 2.62 |
| 231118_at | ankyrin repeat domain 35 | ANKRD35 | chr1q21.1 | 0.33 |
| 231183_s_at | Jagged 1 (Alagille syndrome) | JAG1 | chr20p12.1-p11.23 | 0.44 |
| 231774_at | calsenilin, presenilin binding | CSEN | chr2q21.1 | 2.40 |
| | protein, EF hand transcription | | | |
| | factor | | | |
| 231924_at | Homo sapiens cDNA | — | chr11p15.2 | 0.45 |
| | FLJ10570 fis, clone | | | |
| | NT2RP2003117. | | | |
| 231940_at | zinc finger protein 529 | ZNF529 | chr19q13.13 | 0.64 |
| 232195_at | G protein-coupled receptor | GPR158 | chr10p12.1 | 3.45 |
| | 158 | | | |
| 232322_x_at | START domain containing 10 | STARD10 | chr11q13 | 1.93 |
| 234140_s_at | stromal interaction molecule 2 | STIM2 | chr4p15.2 | 0.48 |
| 234672_s_at | hypothetical protein | FLJ10407 | chr1p32.3 | 0.49 |
| | FLJ10407 | | | |
| 235015_at | zinc finger, DHHC domain | ZDHHC9 | chrXq26.1 | 1.79 |
| | containing 9 | | | |
| 235058_at | Hypothetical protein | FLJ10349 | chr1p36.11 | 0.64 |
| | FLJ10349 | | | |
| 235414_at | zinc finger protein 383 | ZNF383 | chr19q13.12 | 0.48 |
| 235431_s_at | pellino 3 alpha | MGC35521 | chr11q13.2 | 2.20 |
| 235500_at | heterogeneous nuclear | HNRPC | chr14q11.2 | 1.82 |
| | ribonucleoprotein C (C1/C2) | | | |
| 235509_at | hypothetical protein | MGC40214 | chr8q22.1 | 0.37 |
| | MGC40214 | | | |
| 235648_at | zinc finger protein 567 | ZNF567 | chr19q13.12 | 0.47 |
| 235721_at | deltex 3 homolog | DTX3 | chr12q13.3 | 1.67 |
| | (Drosophila) | | | |
| 235759_at | EF hand calcium binding | EFCBP1 | chr8q21.3 | 0.19 |
| | protein 1 | | | |
| 235916_at | yippee-like 4 (Drosophila) | YPEL4 | chr11q12.1 | 2.86 |
| 235940_at | chromosome 9 open reading | C9orf64 | chr9q21.32 | 0.25 |
| | frame 64 | | | |
| 235969_at | hypothetical protein | FLJ33996 | chr12q13.13 | 1.67 |
| | FLJ33996 | | | |
| 238547_at | hypothetical protein | HEXIM2 | chr17q21.31 | 1.58 |
| | MGC39389 | | | |
| 239108_at | Male sterility domain | MLSTD1 | chr12p11.22 | 0.41 |
| | containing 1 | | | |
| 239442_at | KIAA0582 protein | KIAA0582 | chr2p14 | 1.93 |
| 240841_at | insulinoma-associated 2 | INSM2 | chr14q13.2 | 2.38 |
| 241858_at | fucose-1-phosphate | FPGT | chr1p31.1 | 0.40 |
| | guanylyltransferase | | | |
| 242263_at | CGI-100 protein | CGI-100 | chr1pter-q31.3 | 0.57 |
| 242269_at | FLJ42875 protein | FLJ42875 | chr1p36.32 | 0.40 |
| 242429_at | zinc finger protein 567 | ZNF567 | chr19q13.12 | 0.51 |
| 243042_at | FLJ35093 protein | FLJ35093 | chr1p31.1 | 0.55 |
| 244462_at | inc finger protein 224 | ZNF224 | chr19q13.2 | 0.53 |
| 244740_at | hypothetical protein | MGC9913 | chr19q13.43 | 0.64 |
| | MGC9913 | | | |
| 33760_at | peroxisomal biogenesis factor | PEX14 | chr1p36.22 | 0.60 |
| | 14 | | | |
| 38398_at | MAP-kinase activating death | MADD | chr11p11.2 | 1.50 |
| | domain | | | |
| 38710_at | OTU domain, ubiquitin | OTUB1 | chr11q13.1 | 1.44 |
| | aldehyde binding 1 | | | |

TABLE 6
Differentially expressed probesets, which are able to discriminate on
basis of loss of heterozygosity (LOH) on the 19q locus

| | | | | ratio loss/no |
|---|---|---|---|---|
| Probe Set ID | Title | Gene Symbol | location | loss |
| 200650_s_at | lactate dehydrogenase A | LDHA | Chr: 11p15.4 | 0.31 |
| 21058_s_at | chemokine-like factor | CKLF | Chr: 16q22.1 | 0.67 |
| 218624_s_at | hypothetical protein | MGC2752 | Chr: 19p13.2 | 0.56 |
| | MGC2752 | | | |
| 200826_at | small nuclear | SNRPD2 | Chr: 19q13.2 | 0.49 |
| | ribonucleoprotein D2 | | | |
| | polypeptide 16.5 kDa | | | |
| 219603_s_at | zinc finger protein 226 | ZNF226 | Chr: 19q13.2 | 0.35 |
| 222028_at | zinc finger protein 45 (a | ZNF45 | Chr: 19q13.2 | 0.55 |
| | Kruppel-associated box | | | |
| | (KRAB) domain | | | |
| | polypeptide) | | | |
| 229123_at | zinc finger protein 224 | ZNF224 | Chr: 19q13.2 | 0.54 |
| 244462_at | zinc finger protein 224 | ZNF224 | Chr: 19q13.2 | 0.52 |
| 219495_s_at | zinc finger protein 180 | ZNF180 | Chr: 19q13.2 | 0.57 |
| | (HHZ168) | | | |
| 206175_x_at | zinc finger protein 222 | ZNF222 | Chr: 19q13.2 | 0.43 |
| 228131_at | excision repair cross- | ERCC1 | Chr: 19q13.2-q13.3 | 0.51 |
| | complementing rodent | | | |
| | repair deficiency, | | | |
| | complementation group | | | |
| | 1 (includes overlapping | | | |
| | antisense sequence) | | | |
| 201194_at | selenoprotein W, 1 | SEPW1 | Chr: 19q13.3 | 0.48 |
| 225434_at | death effector domain- | DEDD2 | Chr: 19q13.31 | 0.49 |
| | containing DNA binding | | | |
| | protein 2 | | | |
| 227689_at | zinc finger protein 227 | ZNF227 | Chr: 19q13.32 | 0.57 |
| 202153_s_at | nucleoporin 62 kDa | NUP62 | Chr: 19q13.33 | 0.47 |
| 209751_s_at | spondyloepiphyseal | SEDL/SEDLP | Chr: 19q13.4 | 0.54 |
| | dysplasia, late | | | |
| 207753_at | zinc finger protein 304 | ZNF304 | Chr: 19q13.4 | 0.53 |
| 205497_at | zinc finger protein 175 | ZNF175 | Chr: 19q13.4 | 0.62 |
| 1556678_a_at | Homo sapiens full | — | Chr: 19q13.41 | 0.59 |
| | length insert cDNA | | | |
| | clone ZD41C11 | | | |
| 219848_s_at | zinc finger protein 432 | ZNF432 | Chr: 19q13.41 | 0.51 |
| 202408_s_at | PRP31 pre-mRNA | PRPF31 | Chr: 19q13.42 | 0.49 |
| | processing factor 31 | | | |
| | homolog (yeast) | | | |
| 229614_at | hypothetical protein | LOC162967 | Chr: 19q13.42 | 0.62 |
| | LOC162967 | | | |
| 225256_at | Homo sapiens | | Chr: 19q13.43 | 0.55 |
| | transcribed sequence | | | |
| | with weak similarity to | | | |
| | protein | | | |
| | ref: NP_071431.1 | | | |
| | (H. sapiens) cytokine | | | |
| | receptor-like factor 2; | | | |
| | cytokine receptor CRL2 | | | |
| | precusor [Homo | | | |
| | sapiens] | | | |
| 238436_s_at | Homo sapiens | — | Chr: 19q13.43 | 0.64 |
| | transcribed sequences | | | |
| 238437_at | Homo sapiens | — | Chr: 19q13.43 | 0.60 |
| | transcribed sequences | | | |
| 228652_at | hypothetical protein | FLJ38288 | Chr: 19q13.43 | 0.51 |
| | FLJ38288 | | | |
| 244741_s_at | LOC342935 | | Chr: 19q13.43 | 0.61 |
| 244740_at | LOC342935 | | Chr: 19q13.43 | 0.65 |
| 201274_at | proteasome (prosome, | PSMA5 | Chr: 1p13 | 0.60 |
| | macropain) subunit, | | | |
| | alpha type, 5 | | | |
| 211755_s_at | ATP synthase, H+ | ATP5F1 | Chr: 1p13.2 | 0.68 |
| | transporting, | | | |
| | mitochondrial F0 | | | |
| | complex, subunit b, | | | |
| | isoform 1 | | | |
| 224729_s_at | ATP synthase | ATPAF1 | Chr: 1p33 | 0.48 |
| | mitochondrial F1 | | | |
| | complex assembly | | | |
| | factor 1 | | | |
| 218080_x_at | Fas (TNFRSF6) | FAF1 | Chr: 1p33 | 0.51 |
| | associated factor 1 | | | |
| 213622_at | collagen, type IX, alpha 2 | COL9A2 | Chr: 1p33-p32 | 0.38 |
| 203359_s_at | c-myc binding protein | MYCBP | Chr: 1p33-p32.2 | 0.51 |
| 228970_at | archease | ARCH | Chr: 1p34.3 | 0.53 |
| 202139_at | aldo-keto reductase | AKR7A2 | Chr: 1p35.1-p36.23 | 0.58 |
| | family 7, member A2 | | | |
| | (aflatoxin aldehyde | | | |
| | reductase) | | | |
| 212491_s_at | DnaJ (Hsp40) homolog, | DNAJC8 | Chr: 1p35.3 | 0.61 |
| | subfamily C, member 8 | | | |
| 201225_s_at | serine/arginine | SRRM1 | Chr: 1p36.11 | 0.71 |
| | repetitive matrix 1 | | | |
| 224867_at | similar to Putative | | Chr: 1p36.13 | 0.54 |
| | protein of fungal and | | | |
| | metazoan origin (11.1 kD) | | | |
| 212401_s_at | cell division cycle 2-like 2 | CDC2L2 | Chr: 1p36.3 | 0.70 |
| 222000_at | hypothetical protein | LOC339448 | Chr: 1p36.32 | 0.66 |
| | LOC339448 | | | |
| 213340_s_at | KIAA0495 | KIAA0495 | Chr: 1p36.32 | 0.38 |
| 220526_s_at | mitochondrial ribosomal | MRPL20 | Chr: 1p36.3-p36.2 | 0.56 |
| | protein L20 | | | |
| 202297_s_at | RER1 homolog (S. cerevisiae) | RER1 | Chr: 1p36.32 | 0.50 |
| 236369_at | Homo sapiens | — | Chr: 20q11.21 | 1.38 |
| | transcribed sequence | | | |
| | with weak similarity to | | | |
| | protein prf: 2109260A | | | |
| | (H. sapiens) 2109260A | | | |
| | B cell growth factor | | | |
| | [Homo sapiens] | | | |
| 202096_s_at | benzodiazapine | BZRP | Chr: 22q13.31 | 0.39 |
| | receptor (peripheral) | | | |
| 228538_at | similar to Zinc finger | | Chr: 3p21.33 | 0.43 |
| | protein 7 (Zinc finger | | | |
| | protein KOX4) (Zinc | | | |
| | finger protein HF.16) | | | |
| 202763_at | caspase 3, apoptosis- | CASP3 | Chr: 4q34 | 0.51 |
| | related cysteine | | | |
| | protease | | | |
| 201572_x_at | dCMP deaminase | DCTD | Chr: 4q35.1 | 0.28 |
| 210137_s_at | dCMP deaminase | DCTD | Chr: 4q35.1 | 0.18 |
| 201571_s_at | dCMP deaminase | DCTD | Chr: 4q35.1 | 0.28 |
| 233103_at | Homo sapiens cDNA | — | Chr: 5q14.1 | 0.40 |
| | FLJ14109 fis, clone | | | |
| | MAMMA1001322, | | | |
| | moderately similar to B- | | | |
| | CELL GROWTH | | | |
| | FACTOR | | | |
| | PRECURSOR. | | | |
| 203787_at | single-stranded DNA | SSBP2 | Chr: 5q14.1 | 0.45 |
| | binding protein 2 | | | |
| 210829_s_at | single-stranded DNA | SSBP2 | Chr: 5q14.1 | 0.38 |
| | binding protein 2 | | | |
| 210059_s_at | mitogen-activated | MAPK13 | Chr: 6p21.31 | 0.46 |
| | protein kinase 13 | | | |
| 231067_s_at | A kinase (PRKA) | AKAP12 | Chr: 6q24-q25 | 0.55 |
| | anchor protein (gravin) | | | |
| | 12 | | | |
| 203819_s_at | IGF-II mRNA-binding | IMP-3 | Chr: 7p11 | 0.13 |
| | protein 3 | | | |
| 218640_s_at | pleckstrin homology | PLEKHF2 | Chr: 8q22.1 | 0.35 |
| | domain containing, | | | |
| | family F (with FYVE | | | |
| | domain) member 2 | | | |
| 222699_s_at | pleckstrin homology | PLEKHF2 | Chr: 8q22.1 | 0.37 |
| | domain containing, | | | |
| | family F (with FYVE | | | |
| | domain) member 2 | | | |
| 228284_at | transducin-like | TLE1 | Chr: 9q21.32 | 0.50 |
| | enhancer of split 1 | | | |
| | (E(sp1) homolog, | | | |
| | Drosophila) | | | |
| 203222_s_at | transducin-like | TLE1 | Chr: 9q21.32 | 0.39 |
| | enhancer of split 1 | | | |
| | (E(sp1) homolog, | | | |
| | Drosophila) | | | |
| 223398_at | hypothetical protein | MGC11115 | Chr: 9q22.32 | 0.25 |
| | MGC11115 | | | |
| 226809_at | Homo sapiens cDNA | — | Cross Hyb Matching | 0.17 |
| | FLJ30428 fis, clone | | Probes | |
| | BRACE2008941. | | | |

TABLE 7
Differentially expressed probesets, which are able to discriminate on
basis of loss of heterozygosity (LOH) on both the 1p and 19 q loci

| | | | | ratio loss/no |
|---|---|---|---|---|
| Probe Set ID | Title | Gene Symbol | Location | loss |
| 201177_s_at | SUMO-1 activating enzyme | UBA2 | Chr: 19q12 | 0.45 |
| | subunit 2 | | | |
| 215019_x_at | KIAA1827 protein | KIAA1827 | Chr: 19q13 | 0.56 |
| 201258_at | ribosomal protein S16 | RPS16 | Chr: 19q13.1 | 0.55 |
| 226131_s_at | ribosomal protein S16 | RPS16 | Chr: 19q13.1 | 0.71 |
| 212131_at | DKFZP434D1335 protein | DKFZP434D1335 | Chr: 19q13.12 | 0.49 |
| 218490_s_at | zinc finger protein 302 | ZNF302 | Chr: 19q13.12 | 0.50 |
| 219818_s_at | evolutionarily conserved G- | ECGP | Chr: 19q13.12 | 0.57 |
| | patch domain containing | | | |
| 231940_at | KIAA1615 protein | KIAA1615 | Chr: 19q13.13 | 0.60 |
| 235648_at | hypothetical protein | MGC45586 | Chr: 19q13.13 | 0.47 |
| | MGC45586 | | | |
| 219495_s_at | zinc finger protein 180 | ZNF180 | Chr: 19q13.2 | 0.55 |
| | (HHZ168) | | | |
| 206175_x_at | zinc finger protein 222 | ZNF222 | Chr: 19q13.2 | 0.38 |
| 235702_at | Homo sapiens transcribed | — | Chr: 19q13.31 | 0.59 |
| | sequences | | | |
| 205497_at | zinc finger protein 175 | ZNF175 | Chr: 19q13.4 | 0.61 |
| 1556678_a_at | Homo sapiens full length | LOC284371 | Chr: 19q13.41 | 0.58 |
| | insert cDNA clone ZD41C11 | | | |
| 219848_s_at | zinc finger protein 432 | ZNF432 | Chr: 19q13.41 | 0.51 |
| 228652_at | hypothetical protein | FLJ38288 | Chr: 19q13.43 | 0.51 |
| | FLJ38288 | | | |
| 242140_at | similar to envelope protein | LOC113386 | Chr: 19q13.43 | 0.45 |
| 244740_at | LOC342935 | | Chr: 19q13.43 | 0.61 |
| 208374_s_at | capping protein (actin | CAPZA1 | Chr: 1p13.1 | 0.57 |
| | filament) muscle Z-line, | | | |
| | alpha 1 | | | |
| 211755_s_at | ATP synthase, H+ | ATP5F1 | Chr: 1p13.2 | 0.61 |
| | transporting, mitochondrial | | | |
| | F0 complex, subunit b, | | | |
| | isoform 1 | | | |
| 226000_at | hypothetical protein | DKFZp547A023 | Chr: 1p13.2 | 0.48 |
| | DKFZp547A023 | | | |
| 230300_at | Homo sapiens transcribed | — | Chr: 1p13.3 | 0.49 |
| | sequences | | | |
| 222495_at | protein x 013 | AD-020 | Chr: 1p13.3 | 0.52 |
| 223331_s_at | DEAD (Asp-Glu-Ala-Asp) | DDX20 | Chr: 1p21.1-p13.2 | 0.54 |
| | box polypeptide 20 | | | |
| 228661_s_at | Homo sapiens, clone | — | Chr: 1p21.2 | 0.54 |
| | IMAGE: 4821863, mRNA | | | |
| 219939_s_at | NRAS-related gene | D1S155E | Chr: 1p22 | 0.65 |
| 205263_at | B-cell CLL/lymphoma 10 | BCL10 | Chr: 1p22 | 0.56 |
| 209187_at | down-regulator of | DR1 | Chr: 1p22.1 | 0.50 |
| | transcription 1, TBP-binding | | | |
| | (negative cofactor 2) | | | |
| 215017_s_at | hypothetical protein | FLJ20275 | Chr: 1p22.1 | 0.24 |
| | FLJ20275 | | | |
| 218462_at | RNA processing factor 1 | RPF1 | Chr: 1p22.3 | 0.43 |
| 228135_at | gm117 | gm117 | Chr: 1p22.3 | 0.57 |
| 200902_at | 15 kDa selenoprotein | 15-sep | Chr: 1p31 | 0.56 |
| 202502_at | acyl-Coenzyme A | ACADM | Chr: 1p31 | 0.51 |
| | dehydrogenase, C-4 to C-12 | | | |
| | straight chain | | | |
| 212893_at | DKFZP564I052 protein | DKFZP564I052 | Chr: 1p31.1 | 0.49 |
| 208709_s_at | nardilysin (N-arginine | NRD1 | Chr: 1p32.2-p32.1 | 0.63 |
| | dibasic convertase) | | | |
| 223017_at | endoplasmic reticulum | TLP19 | Chr: 1p32.3 | 0.51 |
| | thioredoxin superfamily | | | |
| | member, 18 kDa | | | |
| 218080_x_at | Fas (TNFRSF6) associated | FAF1 | Chr: 1p33 | 0.48 |
| | factor 1 | | | |
| 242086_at | spermatogenesis associated 6 | SPATA6 | Chr: 1p33 | 0.29 |
| 223230_at | hypothetical protein | FLJ14936 | Chr: 1p33-p32.1 | 0.63 |
| | FLJ14936 | | | |
| 213798_s_at | CAP, adenylate cyclase- | CAP1 | Chr: 1p34.2 | 0.57 |
| | associated protein 1 (yeast) | | | |
| 228970_at | archease | ARCH | Chr: 1p34.3 | 0.50 |
| 212491_s_at | DnaJ (Hsp40) homolog, | DNAJC8 | Chr: 1p35.3 | 0.52 |
| | subfamily C, member 8 | | | |
| 235058_at | Homo sapiens transcribed | — | Chr: 1p36.11 | 0.62 |
| | sequence with weak | | | |
| | similarity to protein | | | |
| | ref: NP_060265.1 | | | |
| | (H. sapiens) hypothetical | | | |
| | protein FLJ20378 [Homo | | | |
| | sapiens] | | | |
| 204299_at | FUS interacting protein | FUSIP1 | Chr: 1p36.11 | 0.53 |
| | (serine-arginine rich) 1 | | | |
| 206095_s_at | FUS interacting protein | FUSIP1 | Chr: 1p36.11 | 0.51 |
| | (serine-arginine rich) 1 | | | |
| 224867_at | similar to Putative protein of | | Chr: 1p36.13 | 0.49 |
| | fungal and metazoan origin | | | |
| | (11.1 kD) | | | |
| 202675_at | succinate dehydrogenase | SDHB | Chr: 1p36.1-p35 | 0.68 |
| | complex, subunit B, iron | | | |
| | sulfur (lp) | | | |
| 226532_at | Full-length cDNA clone | — | Chr: 1p36.22 | 0.50 |
| | CS0DD009YD14 of | | | |
| | Neuroblastoma Cot 50- | | | |
| | normalized of Homo sapiens | | | |
| | (human) | | | |
| 222000_at | hypothetical protein | LOC339448 | Chr: 1p36.32 | 0.63 |
| | LOC339448 | | | |
| 214611_at | glutamate receptor, | GRIK1 | Chr: 21q22.11 | 0.39 |
| | ionotropic, kainate 1 | | | |
| 203787_at | single-stranded DNA binding | SSBP2 | Chr: 5q14.1 | 0.42 |
| | protein 2 | | | |
| 231067_s_at | A kinase (PRKA) anchor | AKAP12 | Chr: 6q24-q25 | 0.51 |
| | protein (gravin) 12 | | | |
| 218640_s_at | pleckstrin homology domain | PLEKHF2 | Chr: 8q22.1 | 0.26 |
| | containing, family F (with | | | |
| | FYVE domain) member 2 | | | |
| 222699_s_at | pleckstrin homology domain | PLEKHF2 | Chr: 8q22.1 | 0.30 |
| | containing, family F (with | | | |
| | FYVE domain) member 2 | | | |
| 202241_at | phosphoprotein regulated by | C8FW | Chr: 8q24.13 | 0.32 |
| | mitogenic pathways | | | |
| 223796_at | cell recognition molecule | CASPR3 | Chr: 9p12 | 0.41 |
| | CASPR3 | | | |
| 203222_s_at | transducin-like enhancer of | TLE1 | Chr: 9q21.32 | 0.38 |
| | split 1 (E(sp1) homolog, | | | |
| | Drosophila) | | | |
| 223398_at | hypothetical protein | MGC11115 | Chr: 9q22.32 | 0.19 |
| | MGC11115 | | | |
| 229498_at | Homo sapiens transcribed | MRNA; cDNA | Chr: Xq26.2 | 0.26 |
| | sequences | DKFZp779M2422 | | |
| | | (from clone | | |
| | | DKFZp779M2422) | | |
| 226411_at | similar to ecotropic viral | LOC115704 | Chr: 19p13.3 | 2.24 |
| | integration site 5; | | | |
| | Neuroblastoma stage 4S | | | |
| | gene | | | |

1-21. (canceled)

22. A method for producing a classification scheme for oligodendroglial tumors comprising the steps of:
a) providing a plurality of reference samples, said reference samples comprising cell samples from a plurality of reference subjects suffering from oligodendroglial tumors, with known responsiveness to therapy and survival or with known loss of heterozygosity of 1p or 19q;
b) providing reference profiles by establishing a gene expression profile, matched with parameters for treatment sensitivity, survival and loss of heterozygosity for each of said reference samples individually;
c) clustering said individual reference profiles according to a statistical procedure, comprising:
(i) K-means clustering,
(ii) hierarchical clustering, and
(iii) Pearson correlation coefficient analysis; and
d) assigning an oligodendroglial tumor class according to treatment sensitivity, survival or loss of heterozygosity to each cluster.

23. The method according to claim 22, wherein the clustering of said gene expression profiles is performed based on the information of differentially-expressed genes and the treatment sensitivity, survival or loss of heterozygosity of the subject.

24. The method according to claim 22, wherein the clustering of said gene expression profiles with respect to treatment response is performed based on the information of the genes of Table 3.

25. The method according to claim 22, wherein the clustering of said gene expression profiles with respect to survival is performed based on the information of the genes of Table 4.

26. The method according to claim 22, wherein the clustering of said gene expression profiles with respect to loss of heterozygosity of 1p is performed based on the information of the genes of Table 5.

27. The method according to claim 22, wherein the clustering of said gene expression profiles with respect to loss of heterozygosity of 19q is based on the information of the genes of Table 6.

28. The method according to claim 22, wherein the clustering of said gene expression profiles with respect to loss of heterozygosity of 1p and 19q is performed based on the information of the genes of Table 7.

29. A method for classifying an oligodendroglial tumor of a subject suffering from oligodendroglial tumor, comprising the steps of:
a) providing a classification scheme for oligodendroglial tumors according to the method of claim 22;
b) providing a subject profile by establishing a gene expression profile for said subject;
c) clustering the subject profile together with a plurality of reference profiles;
d) determining in said scheme the clustered position of said subject profile among the reference profiles; and
e) assigning an oligodendroglial tumor class that corresponds to said clustered position to said oligodendroglial tumor.

30. The method according to claim 29, wherein said gene expression profile with respect to treatment response comprises a plurality of expression parameters of a set of genes according to Table 3.

31. The method according to claim 29, wherein said gene expression profile with respect to survival comprises a plurality of expression parameters of a set of genes according to Table 4.

32. The method according to claim 29, wherein said gene expression profile with respect to 1p loss of heterozygosity comprises a plurality of expression parameters of a set of genes according to Table 5.

33. The method according to claim 29, wherein said gene expression profile with respect to 19q heterozygosity comprises a plurality of expression parameters of a set of genes according to Table 6.

34. The method according to claim 29, wherein said gene expression profile with respect to 1p and 19q loss of heterozygosity comprises a plurality of expression parameters of a set of genes according to Table 7.

35. A method of determining the prognosis for a subject suffering from an oligodendroglial tumor, said method comprising the steps of:
a) providing a classification scheme for oligodendroglial tumors by the method according to claim 22;
b) determining a prognosis for each olidendroglial tumor class in said classification scheme based on clinical records for the subjects comprised in said class;
c) establishing an oligodendroglial class of a subject suffering from an oligodendroglial tumor by classifying the oligodendroglial tumor in said subject according to the method of claim 29; and
d) assigning to said subject the prognosis corresponding to the established oligodendroglial tumor class of said subject.

36. A method of determining the prognosis for a subject suffering from an oligodendroglial tumor, said method comprising the steps of:
a) isolating an RNA from tumor cells of said subject;
b) preparing an antisense, biotinylated RNA to said RNA of step a);
c) hybridizing said antisense to said RNA;
d) normalizing a plurality of measured values for a gene set of Table 3;
e) clustering the obtained data together with the reference data, obtained from a reference set of patient with known prognosis; and
f) determining the prognosis on basis of the cluster to which the data of the subject are clustering.

37. An oligonucleotide microarray of maximal 500 probesets, comprising at least 1 oligonucleotide probe capable of hybridizing under stringent conditions to a gene of an oligodendroglial tumor-associated genes selected from Tables 3-7.

38. The oligonucleotide microarray of maximal 500 probesets of claim 37, wherein the probesets comprise at least 2 oligonucleotide probes.

39. The oligonucleotide microarray of maximal 500 probesets of claim 37, wherein the probesets comprise at least 25 oligonucleotide probes.

40. The oligonucleotide microarray of maximal 500 probesets of claim 37, wherein the probesets comprise at least 100 oligonucleotide probes.

41. A kit comprising an oligonucleotide microarray according to claim 37 and means for comparing a gene expression profile determined by using said microarray with a database of oligodendroglial tumor reference expression profiles.
